(19) United States
(12) Patent Application Publication
(10) Pub. No.: US 2026/0356915 A1
(43) Pub. Date: Nov. 12, 2026

(54) FAS LIGAND VARIANT AND RECOMBINANT CELL HAVING INCREASED CYTOTOXICITY AND GREATER SURVIVAL

(71) Applicant: Onni Biotechnologies Oy, Helsinki (FI)

(72) Inventors: Xenia Glukhova, Helsinki (FI); Igor Beletsky, Helsinki (FI)

(21) Appl. No.: 19/488,597

(22) PCT Filed: May 28, 2024
(86) PCT No.: PCT/FI2024/050277

§ 371 Date: Nov. 27, 2025

(30) Foreign Application Priority Data
May 29, 2023 (FI) 20237105

(51) Int. Cl. C07K 14/705; A61K 40/15; A61K 40/42; A61K 40/50; C12N 5/0783; C12N 15/85

(52) U.S. Cl. CPC C07K 14/70575; A61K 40/15; A61K 40/4202; A61K 40/50; C12N 5/0646; C12N 15/85; C12N 2510/00

(57) ABSTRACT

Herein is disclosed variants of Fas ligand (APTL; FASL; CD178; CD95L; ALPS1B; CD95-L; TNFSF6; TNLG1A; APT1LG1) and vectors providing thereof. Further, herein is disclosed a recombinant cell, such as NK cell (natural killer cell), comprising genetic elements allowing production of at least one FasL variant, to increase the cytotoxicity and survival of the recombinant cell, for improving therapeutic activity of the recombinant cell. Also a pharmaceutical composition comprising the recombinant cell and methods for obtaining thereof, as well as NK cells modified to express the Fas ligand variants for use in immunotherapy are disclosed

### TECHNICAL FIELD

The present invention generally relates to Fas ligand (FasL) variants and recombinant cells expressing thereof. The invention relates particularly, though not exclusively, to immune cells, such as natural killer cells expressing FasL variants. The present invention, in some embodiments thereof, relates to methods for improving the functionality of NK cell, such as their cytotoxic activity and survival, to be used in immunotherapy. In particular, these methods comprise a composition of DNA fragments of Fas-ligand (APTL; FASL; CD178; CD95L; ALPS1B; CD95-L; TNFSF6; TNLG1A; APT1LG1) variant and methods of producing recombinant NK cells with increased Fas-ligand production. The invention encompasses also compositions of engineered recombinant NK cells and NK cell lines and uses thereof for treating or for preventing cancer and other immune related disorders. The present disclosure further encompasses natural killer (NK) cells modified to express Fas ligand variants for use in therapy, such as immunotherapy.

### BACKGROUND

This section illustrates useful background information without admission of any technique described herein representative of the state of the art. NK cells are the main cellular effectors of the innate immune system, destroying various targets, including infected or transformed cells, and in some cases senescent or stressed cells (Shimasaki et al., Nat Rev Drug Discov. 2020 March; 19(3):200-2181; Giannoula et al., Biomed J. 2023 Feb. 4:S2319-4170(23) 00005-7). NK cells do not require prior antigen exposure or MHC restriction (Schattner, Duggan. Am J Hematol. 1985 April; 18(4):435-43; Brix et al. U.S. Pat. No. 10,030,065B2/2018). NK cells lack surface T cell receptors (TCRs) and do not induce graft-versus-host disease (GVHD). As such, they are considered as turnkey (“off-shelf”) cell therapy product that can be prepared in advance, optimized, and administered to patients. These attributes endow NK cells with unique advantages for autologous as well as allogeneic therapeutic applications.

NK cell functions, including cytotoxicity, cytokine synthesis, and degranulation, are regulated by signals mediated by inhibitory receptors (particularly killer Ig-like receptors (KIRs) and heterodimeric C-type lectin receptor (NKG2A)), and activating receptors (particularly natural cytotoxicity (NCRs) NKp46, NKp30, NKp44 and lectin-like C-type activator immunoreceptor NKG2D7) that recognize ligands on target cells (Toledo et al., Sci Adv. 2021 Jun. 11; 7(24): eabc16405-8; Rascle et al., Front Immunol. 2023 Jan. 20; 14:1087155; Valton et al., JP2022101530A/2022; Andre, Kübler, ES2772307T3/2020).

NK cells can directly kill tumor cells by a) releasing cytoplasmic granules containing perforin and granzyme, and b) expressing TNF family proteins such as FasL or TRAIL, which induce tumor cell apoptosis by interacting with their respective receptors. Immature NK cells likely use TRAIL-dependent cytotoxicity rather than FasL- or granule release-dependent cytotoxicity, while mature NK cells mainly use the latter two (Zamai et al., J Exp Med. 1998 Dec. 21; 188(12):2375-80). In addition, antibody-dependent cellular cytotoxicity (ADCC) mediated by the CD16 Fc receptor can cause NK-mediated death of target cells that have interacted with antibodies (Bunting et al., Sci Adv. 2022 Mar. 18; 8(11): eabk3327; Orange, Nat Rev Immunol. 2008 September; 8(9):713-25). Besides, IFN-γ produced by activated NK cells also affects the tumor, since IFN-γ induces remodeling of the tumor microenvironment, inhibits tumor angiogenesis, and has antimetastatic activity (von Locquenghien et al., J Clin Invest. 2021 Jan. 4; 131(1):e143296; Granzin et al., Front Immunol. 2017 Apr. 26; 8:458; Chawla-Sarkar et al., Apoptosis. 2003 June; 8(3):237-49; JP7204643B2/2023).

In NK cells, FasL is stored in secretory lysosomes, which also contain granzymes and perforin. The co-localization of FasL, perforin, and granzymes in the same subcellular structures implies that the simultaneous delivery of these apoptosis-inducing molecules to the immunological synapse between effector and target cells can lead to more efficient and faster killing of target cells. The intracellular accumulation of FasL is tightly regulated by its cytoplasmic tail and interacting molecules (Bossi et al., Nat Med. 1999 January; 5(1):90-6; Glukhova et al., Cell Death Dis. 2018 Jan. 22; 9(2):73). When NK cells collide with target cells, such as tumor cells, cytoplasmic granules (secretory lysosomes) comprising FasL are transported to the site of intercellular contact, where they fuse with the plasma membrane, thereby exposing FasL within the immunological synapse. The selective association with lipid rafts on the cell surface increases the death-promoting activity of FasL (Lettau et al., Curr Med Chem. 2008; 15(17):1684-96; Kassahn et al., Cell Death Differ. 2009 January; 16(1):115-24).

It has been shown for T cells that after antigenic stimulation they undergo clonal expansion, rapidly increasing in number, after which their number decreases as a result of programmed cell death. This secondary phase is known as peripheral deletion and is due to the engagement of apoptosis pathways during signaling for clonal expansion. This phenomenon has been termed “activation-induced cell death” (AICD) and is mediated by the interaction of Fas and FasL on activated T cells. Upon antigen stimulation, T cells up-regulate FasL and increase their sensitivity to Fas-mediated apoptosis. The number of T cells declines as Fas-FasL interactions between T cells cause their cell death (Yamauchi et al., Blood. 1996 Jun. 15; 87(12):5127-35; Hennessy et al., J Leukoc Biol. 2019 June; 105(6):1341-1354). A similar phenomenon was also shown for NK cells. During the immune response, the expanded activated NK cells come to express Fas and eventually undergo AICD mediated by their own secretion of FasL (Masuda et al., Cancer Sci. 2020 March; 111(3):807-816; Lopez-Vergès et al., Blood. 2010 Nov. 11; 116(19):3865-74; Lee et al., Cytokine. 2012; 59(3): 547).

Methods and compositions of modifying AICD of T cells and/or NK cells has been previously implicated for diabetes treatment and for anti-cancer therapy, they are summarized below: U.S. Pat. No. 9,624,469B2/2017 (Regulatory immune cells with enhanced targeted cell death effect) discloses that targeted simulation of the process of activation-induced cell death (AICD) at the site of inflammation ameliorate inflammatory insulitis. Inventors have generated regulatory T cells (Tregs) with enhanced cell death effect by chemically attaching to the surface of these cells a chimeric Fas-ligand (FasL) protein and use them for suppression of diabetogenic effector cells at the site of inflammation and for diabetes treatment. These results substantiate the value of modified Treg cells overexpressing a death molecule, such as FasL, for treatment of immune related diseases.

Patent application WO2019014684A1/2019 provides methods of inhibiting AICD of T cells and/or NK cells in a subject with chronic lymphocytic leukemia comprising administering to the subject an inhibitor of interleukin-2 inducible T cell kinase (ITK), ibrutinib. The data demonstrate that ibrutinib therapy is contemplated as an cellular immune modulating agent for CLL and potentially other types of hematologic and solid cancers.

Authors of Australian patent application AU2019347873A1/2021 publish methods of obtaining genetically modified immunoresponsive cells (e.g., T cells or NK cells) comprising the antigen-recognizing receptor and the dominant negative Fas polypeptide. Such T or NK cells are endowed with augmented and selective cytolytic activity at the tumor site.

US Patent application US20190038671A1/2019 discloses the pharmaceutical compositions based on the engineered mammalian cells containing a vector comprising the heterologous nucleic acid encoding the immunomodulator (an immune checkpoint inhibitors or immunoactivators) and a second heterologous nucleic acid encoding the CAR or the TCR. Such T cells can be more resistant to activation-induced cell death and can be widely applicable in cancer immunotherapy.

US Pat. application No. US20210246423A1/2021 proposes methods for improving in vitro expansion and activation of immune cells and preventing AICD. It is based on the discovery that activation of CAR expressed (e.g., transiently expressed) on the surface of immune effector cells provides an effective means to expand and/or activate a population of immune effector cells.

Canadian patent CA2706445C/2019 describes methods for protecting immune cells from cell death with IRX-2. IRX-2, also known as “citoplurikin”, is a leukocyte-derived, natural primary cell derived biologic produced by mononuclear cells stimulated by phytohemagglutinin and ciprofloxacin. IRX-2 protects activated T cells from both extrinsic apoptosis and intrinsic metabolic apoptosis and enhance their anti-tumor activity.

An excess of Fas-ligand on the plasma membrane (in lipid rafts) can lead to the death of cells producing it. Preservation of the Fas-ligand inside the cell, in particular, in secretory lysosomes, is both a protective mechanism and one of the key factors in the cytotoxic activity of the NK cell (Krzewski et al., Front Immunol. 2012 Nov. 9; 3:335; Lee et al., Immun Inflamm Dis. 2018 June; 6(2):312-321). Known “trafficking domains”, primarily the LAMP lumenal domain, effectively target proteins containing them to lysosomal vesicles. Methods of modifying the protein for targeting of the protein to the endosomal/lysosomal compartment are summarized below.

US patent U.S. Pat. No. 5,633,234A/1997 (Lysosomal targeting of immunogens. Expired) discloses a targeting signal that directs proteins to the endosomal/lysosomal compartment. Authors demonstrated that chimeric proteins containing a cytoplasmic endosomal/lysosomal targeting signal will effectively target antigens to that compartment.

US patent US20040157307A1/2004 (Chimeric vaccines) describes a chimeric protein comprising an antigen sequence and a domain for trafficking the protein to an endosomal compartment, irrespective of whether the antigen is derived from a membrane or non-membrane protein. In one preferred aspect of the invention, the trafficking domain comprises a lumenal domain of a LAMP polypeptide.

US patent U.S. Pat. No. 9,993,546B2/2018 (Lysosomal targeting of antigens employing nucleic acids encoding lysosomal membrane polypeptide/antigen chimeras) discloses lysosomal targeting of antigens employing nucleic acids encoding lysosomal membrane polypeptide/antigen chimeras. In one preferred aspect, the trafficking domain comprises a lumenal domain of a LAMP polypeptide. Alternatively, or additionally, the chimeric protein comprises a trafficking domain of an endocytic receptor (e.g., such as DEC-205 or gp200-MR6).

Australian patent AU2019250227B2/2021 (Nucleic acids for treatment of allergies) provides DNA vaccines for the treatment of allergies. The vaccines comprise the coding sequence for one or more allergenic epitopes, and preferably the full protein sequence, of the allergenic protein from which the epitope(s) is derived, fused inframe with the lumenal domain of the lysosomal associated membrane protein (LAMP) and the targeting sequence of LAMP.

The ability to regulate AICD in NK cells has important implications to NK cell therapy for cancer. A common disadvantage to known AICD-modifying techniques is that a) it difficult to control persistent side effects caused by these treatments, and b) AICD inhibition results in greater NK cell survival but less cytotoxicity, and vice versa, increased AICD causes poor survival and higher cytotoxic activity.

Thus, there is a need for alternative and preferably improved human NK cells, with a greater cytotoxicity and more pronounced survival.

### PURPOSE OF THE INVENTION

FasL variants and recombinant cells expressing the FasL variants, offer a significant advantage in immunotherapy by redistributing FasL variants to secretory lysosomes but not to the cell membrane of the recombinant cells, such as recombinant NK cells. This reduces AICD during NK cell activation and, consequently, increases survival and enhances cytotoxic activity of the recombinant cells through release of extracellular vesicles comprising FasL variants by the secretory lysosomes (cytotoxic granules) from the recombinant cells of the invention. In addition, the use of said FasL variants makes it possible to obtain a greater yield of recombinant cells, such as recombinant NK cells with high cytotoxic activity during in vitro cultivation.

### SUMMARY

The appended claims define the scope of protection. Any examples and technical descriptions of apparatuses, products and/or methods in the description and/or drawings not covered by the claims are presented not as embodiments of the invention but as background art or examples useful for understanding the invention. It should be understood that the disclosure is not limited in its application to the details set forth in the following embodiments, the claims, the description, and the figures. The invention is capable of other embodiments and may be practiced or carried out in a variety of other ways.

To address this need for more efficient destruction of cancer cells, there are provided for herein nucleotide and amino acids sequences and vectors that encode genetic constructions that confer both greater survival and increased cytotoxicity on natural killer cells. A further object is to provide methods for producing modified (recombinant) NK cells with increased Fas-ligand (variant) production, compositions containing the cells and uses of said compositions in the treatment of cancers.

According to an aspect of some embodiments of the NK cell is derived from umbilical cord blood, peripheral blood, bone marrow, CD34+ cells, iPSCs or ESC. In some respect the NK cell is human NK cell lines, e. g. NKL (CVCL_0466), YTS (CVCL_D324), NK 3.3 (CVCL_7994), NK-92 (CVCL_2142), KHYG-1 (CVCL_2976), haNK (CVCL_IM23), IaNK (CVCL_VN54) and others. According to some embodiments of the invention, NK cells are cells infiltrated into tissues.

According to a first example aspect, is provided the Fas Ligand (FasL) variant having an intracellular domain comprising at least one amino acid sequence GYXXφ, wherein (X) is any amino acid, and (φ) is an amino acid selected from an amino acid L, I or V, and wherein the amino acid positions of the amino acid sequence correspond to the amino acid positions of the SEQ ID NO: 1.

In an embodiment, the intracellular domain of the Fas Ligand (FasL) variant comprises at least two, preferably at least three amino acid sequences GYXXφ, wherein (X) is any amino acid, and (φ) is an amino acid selected from an amino acid L, I or V, and wherein the amino acid positions of the amino acid sequence correspond to the amino acid positions of the SEQ ID NO: 1. In an embodiment, the intracellular domain of the Fas Ligand (FasL) variant corresponds to the amino acids 1-80 of SEQ ID NO: 1, and wherein said intracellular domain comprises at least one amino acid sequence GYXXφ, wherein (X) is any amino acid, and (φ) is an amino acid selected from an amino acid L, I or V, and wherein the amino acid positions of the amino acid sequence correspond to the amino acid positions of the SEQ ID NO: 1.

The intracellular domain of the wild type FasL constitutes amino acids 1-80, corresponding to the amino acids 1-80 of SEQ ID NO: 47. The intracellular domain of the wild type human FasL constitutes amino acids ¹M-G⁸⁰, corresponding to the amino acids 1-80 of SEQ ID NO: 47. The wild type human FasL has the amino acid sequence of SEQ ID NO: 47.

In an embodiment, the Fas Ligand (FasL) variant has an intracellular domain comprising an amino acid sequence ⁶GYXXφ¹⁰, wherein (X) is any amino acid, and (φ) is an amino acid selected from an amino acid L, I or V, and wherein the amino acid positions of the amino acid sequence correspond to the amino acid positions of the SEQ ID NO: 1.

In an embodiment, the Fas Ligand (FasL) variant has an intracellular domain comprising at least one amino acid sequence selected from ⁶GYXXφ¹⁰, ⁸GYXXφ¹², and ⁶⁷GYXXφ⁷¹, wherein (X) is any amino acid, and (φ) is an amino acid selected from an amino acid L, I or V, and wherein the amino acid positions of the amino acid sequence correspond to the amino acid positions of the SEQ ID NO: 1.

In an embodiment, the FasL variant is obtained by introducing amino acid substitutions to a wild type FasL amino acid sequence. In an embodiment, the FasL variant is obtained by introducing at least four amino acid substitutions to the intracellular domain of the wild type FasL sequence of SEQ ID NO: 47. According to some embodiments of the invention, amino acid substitutions for the modified forms of the Fas-ligand were chosen in accordance with the data of Bonifacino and Traub (Annu Rev Biochem. 2003; 72:395-447). The selected amino acid substitutions are designed to redistribute Fas-ligand transport towards the intracellular depot, including to the secretory lysosomes, but not to the plasma membrane.

In an embodiment, the FasL variant comprises an intracellular domain comprising an amino acid sequence YXXφ, wherein Y is tyrosine, X is any amino acid, and φ is a hydrophobic amino acid selected from leucine, isoleucine or valine (L; I; V).

According to some embodiments of the invention, the YXXφ site in the intracellular domain of the Fas-ligand is involved in interaction with proteins of the adapter protein (AP) complex and is responsible for the internalization of the ligand and its transport into secretory lysosomes. In some embodiments, glycine residue preceding tyrosine promotes protein transport into the lysosomal compartment.

In an embodiment, the FasL variant comprises an intracellular domain comprising an amino acid sequence GYXXφ, wherein G is Glycine, Y is tyrosine, X is any amino acid, and φ is a hydrophobic amino acid selected from leucine, isoleucine or valine (L; I; V).

According to some embodiments of the invention, modified forms of the Fas-ligand comprise amino acid substitutions resulting in GYXXφ repeat sites, where X is any amino acid, ¢ is a hydrophobic amino acid (leucine, isoleucine or valine (L; I; V)), that promotes their transport to secretory lysosomes.

In an embodiment, the intracellular domain of the FasL variant comprises an amino acid sequence selected from GYXXL, GYXXI or GYXXV, wherein X is any amino acid.

In an embodiment, the FasL variant is FasLmod1. According to some embodiments of the invention, the modified form of the Fas-ligand, FasLmod1, contains the following sites that facilitate their transportation to secretory lysosomes and increases cytotoxicity: ⁶GYGYL¹⁰, ⁸GYLQI¹², ¹³YWVL¹⁶, where G—glycine, Y—tyrosine, L—leucine, Q—glutamine, I—isoleucine, W—tryptophan, V—valine, P—Proline.

In an embodiment, the FasL variant comprises an intracellular domain comprising at least one amino acid sequence selected from ⁶GYGYL¹⁰, ⁸GYLQI¹², and ¹³YWVL¹⁶, wherein the amino acid positions correspond to the amino acid positions of the SEQ ID NO: 1. In an embodiment, the FasL variant comprises an intracellular domain comprising an amino acid sequence ⁶GYGYLQIYWVL¹⁶ wherein the amino acid positions correspond to the amino acid positions of the SEQ ID NO: 1.

In an embodiment, the FasL variant is FasLmod2. According to some embodiments of the invention, the modified form of the Fas-ligand, FasLmod2, contains the following sites that facilitate their transportation to secretory lysosomes and increases cytotoxicity:

⁶GYGYI¹⁰, ⁸GYIQI¹², ¹³YWVI¹⁶, where amino acid one letter code is as presented previously.

In an embodiment, the FasL variant comprises an intracellular domain comprising at least one amino acid sequence selected from ⁶GYGYI¹⁰, ⁸GYIQI¹², and ¹³YWVI¹⁶, wherein the amino acid positions correspond to the amino acid positions of the SEQ ID NO: 1. In an embodiment, the FasL variant comprises an intracellular domain comprising an amino acid sequence ⁶GYGYIQIYWVI¹⁶, wherein the amino acid positions correspond to the amino acid positions of the SEQ ID NO: 1.

In an embodiment, the FasL variant is FasLmod3. According to some embodiments of the invention, the modified form of the Fas-ligand, FasLmod3, contains the following sites that facilitate their transportation to secretory lysosomes and increases cytotoxicity:
⁶GYGYV¹⁰, ⁸GYVQI¹², ¹³YWVV¹⁶, where amino acid one letter code is as presented previously.

In an embodiment, the FasL variant comprises an intracellular domain comprising at least one amino acid sequence selected from ⁶GYGYV¹⁰, ⁸GYVQI¹², and ¹³YWVV¹⁶, wherein the amino acid positions correspond to the amino acid positions of the SEQ ID NO: 1. In an embodiment, the FasL variant comprises an intracellular domain comprising an amino acid sequence ⁶GYGYVQIYWVV¹⁶, wherein the amino acid positions correspond to the amino acid positions of the SEQ ID NO: 1.

In an embodiment, the FasL variant is FasLmod4. According to some embodiments of the invention, the modified form of the Fas-ligand, FasLmod4, contains the following sites that facilitate their transportation to secretory lysosomes and increases cytotoxicity:
⁶GYGYL¹⁰, ⁸GYLQI¹², ¹³YWVL¹⁶, ⁶⁷GYPPL⁷¹, where amino acid one letter code is as presented previously.

In an embodiment, the FasL variant comprises an intracellular domain comprising at least one amino acid sequence selected from ⁶GYGYL¹⁰, ⁸GYLQI¹², ¹³YWVL¹⁶, and ⁶⁷GYPPL⁷¹, wherein the amino acid positions correspond to the amino acid positions of the SEQ ID NO: 1. In an embodiment, the FasL variant comprises an intracellular domain comprising amino acid sequences ⁶GYGYLQIYWVL¹⁶ and ⁶⁷GYPPL⁷¹, wherein the amino acid positions correspond to the amino acid positions of the SEQ ID NO: 1.

In an embodiment, the FasL variant is FasLmod5. According to some embodiments of the invention, the modified form of the Fas-ligand, FasLmod5, contains the following sites that facilitate their transportation to secretory lysosomes and increases cytotoxicity: ⁶GYGYI¹⁰, ⁸GYIQI¹², ¹³YWVI¹⁶, ⁶⁷GYPPI⁷¹, where amino acid one letter code is as presented previously.

In an embodiment, the FasL variant comprises an intracellular domain comprising at least one amino acid sequence selected from ⁶GYGYI¹⁰, ⁸GYIQI¹², ¹³YWVI¹⁶, and ⁶⁷GYPPI⁷¹, wherein the amino acid positions correspond to the amino acid positions of the SEQ ID NO: 1. In an embodiment, the FasL variant comprises an intracellular domain comprising amino acid sequences ⁶GYGYIQIYWVI¹⁶ and ⁶⁷GYPPI⁷¹, wherein the amino acid positions correspond to the amino acid positions of the SEQ ID NO: 1.

In an embodiment, the FasL variant is FasLmod6. According to some embodiments of the invention, the modified form of the Fas-ligand, FasLmod6, contains the following sites that facilitate their transportation to secretory lysosomes and increases cytotoxicity:
⁶GYGYV¹⁰, ⁸GYVQI¹², ¹³YWVV¹⁶, ⁶⁷GYPPV⁷¹, where amino acid one letter code is as presented previously.

In an embodiment, the FasL variant comprises an intracellular domain comprising at least one amino acid sequence selected ⁶GYGYV¹⁰, ⁸GYVQI¹², ¹³YWVV¹⁶, and ⁶⁷GYPPV⁷¹, wherein the amino acid positions correspond to the amino acid positions of the SEQ ID NO: 1. In an embodiment, the FasL variant comprises an intracellular domain comprising amino acid sequences ⁶GYGYVQIYWVV¹⁶ and ⁶⁷GYPPV⁷¹ wherein the amino acid positions correspond to the amino acid positions of the SEQ ID NO: 1.

In an embodiment, the intracellular domain of the FasL variant comprises an amino acid sequence selected from ⁶GYGYL¹⁰, ⁶GYGYI¹⁰, or ⁶GYGYV¹⁰, wherein the amino acid positions of the sequence correspond to the amino acid positions of the SEQ ID NO: 1. In an embodiment, the intracellular domain of the FasL variant comprises an amino acid sequence ⁶GYXXφ¹⁰, wherein X is any amino acid and φ is an amino acid selected from an amino acid L, I or V; wherein the amino acid sequence of the FasL variant has at least 80%, preferably at least 85%, more preferably at least 90%, most preferably at least 95% sequence identity with the sequence of SEQ ID NO: 1, and wherein the amino acid positions of the sequence correspond to the amino acid positions of the SEQ ID NO: 1. In an embodiment, the intracellular domain of the FasL variant comprises an amino acid sequence GYXXφ, wherein X is any amino acid and φ is an amino acid selected from an amino acid L, I or V; wherein the amino acid sequence of the FasL variant has at least 80%, preferably at least 85%, more preferably at least 90%, more preferably at least 95%, most preferably at least 97% sequence identity with the amino acids 1-80 of the SEQ ID NO: 1, and wherein the amino acid positions of the sequence correspond to the amino acid positions of the SEQ ID NO: 1. In such an embodiment, the amino acid sequence GYXXφ is preferably located at least at ⁶GYXXφ¹⁰ the amino acid positions corresponding to the amino acid positions of the SEQ ID NO: 1. In an embodiment, the intracellular domain of the FasL variant comprises amino acid substitutions at least at amino acid positions N⁶, P⁸, P¹⁰ and D¹⁶, wherein the amino acid positions correspond to the wild type FasL amino acids of the SEQ ID NO:47.

In an embodiment, the intracellular domain of the FasL variant comprises a set of amino acid substitutions at amino acid positions selected from:
P⁸, and P¹⁰;
N⁶, P⁸, and P¹⁰;
N⁶, P⁸, P¹⁰, and D¹⁶;
N⁶, P⁸, P¹⁰, I¹⁶, P⁶⁷, and L⁶⁸;
N⁶, P⁸, P¹⁰, I¹⁶, P⁶⁷, L⁶⁸, L⁷¹ and
combinations thereof,
   wherein the amino acid positions correspond to the wild type FasL amino acids of the SEQ ID NO: 47. In an embodiment, the intracellular domain of the FasL variant comprises amino acid substitutions at least at positions N6G, P8G, P10φ and D16Z, wherein both (φ) and (Z) are independently selected from amino acids L, I or V, and wherein the amino acid positions correspond to the wild type FasL amino acids of the SEQ ID NO:47.

In an embodiment, the intracellular domain of the FasL variant comprises a set of amino acid substitutions at amino acid positions selected from:
P8G, and P10L; or P8G, and P10I; or P8G, and P10V; or
N6G, P8G, and P10L; or N6G, P8G, and P10I; or N6G, P8G, and P10V; or
N6G, P8G, P10L, and D16I; or N6G, P8G, P10I, and D16I; or N6G, P8G, P10V, and D16I; or N6G, P8G, P10L, and D16V; or N6G, P8G, P10I, and D16V; or N6G, P8G, P10V, and D16V; or N6G, P8G, P10L, and D16L; or N6G, P8G, P10I, and D16L; or N6G, P8G, P10V, and D16L; or
P67G, and L68Y; or P67G, L68Y and L71I; or P67G, L68Y and L71V, and
combinations thereof,
   wherein the amino acid positions correspond to the wild type FasL amino acids of the SEQ ID NO: 47. In an embodiment, the intracellular domain of the FasL variant comprises an amino acid sequence ⁶GYGYφQIYWVZ¹⁶, wherein both, (φ) and (Z), are selected independently from an amino acid L, I or V, and wherein the amino acid positions of the amino acid sequence correspond to the amino acid positions of the SEQ ID NO: 1. In such an embodiment, the amino acids at the positions X¹⁰ and X¹⁶ can be selected independently.

In an embodiment, the intracellular domain of the FasL variant comprises an amino acid sequence ⁶GYGYLQIYWVL¹⁶, or ⁶GYGYIQIYWVI¹⁶, or ⁶GYGYVQIYWVV¹⁶, and wherein the amino acid positions correspond to the amino acid positions of the SEQ ID NO: 1. In an embodiment, the intracellular domain of the FasL variant comprises an amino acid sequence ⁶GYGYLQIYWVL¹⁶, or ⁶GYGYIQIYWVI¹⁶, or ⁶GYGYVQIYWVV¹⁶, or ⁶GYGYLQIYWVL¹⁶ and ⁶⁷GYPPL⁷¹, or ⁶GYGYIQIYWVI¹⁶ and ⁶⁷GYPPI⁷¹, or ⁶GYGYVQIYWVV¹⁶ and ⁶⁷GYPPV⁷¹, and wherein the amino acid positions correspond to the amino acid positions of the SEQ ID NO: 1.

In an embodiment, the intracellular domain of the FasL variant comprises an amino acid sequence ⁶GYGYLQIYWVL¹⁶, ⁶GYGYLQIYWVI¹⁶, ⁶GYGYLQIYWVV¹⁶, ⁶GYGYIQIYWVL¹⁶, ⁶GYGYIQIYWVI¹⁶, ⁶GYGYIQIYWVV¹⁶, ⁶GYGYVQIYWVL¹⁶, ⁶GYGYVQIYWVI¹⁶, or ⁶GYGYVQIYWVV¹⁶, wherein the amino acid positions of the sequence correspond to the amino acid positions of the SEQ ID NO: 1. In an embodiment, the FasL variant comprises an intracellular domain comprising an amino acid sequence ⁶GYGYLQIYWVL¹⁶, or ⁶GYGYIQIYWVI¹⁶, or ⁶GYGYVQIYWVV¹⁶, wherein the amino acid positions correspond to the amino acid positions of the SEQ ID NO: 1.

In an embodiment, the FasL variant comprises an intracellular domain comprising an amino acid sequence ⁶GYGYLQIYWVL¹⁶, or ⁶GYGYIQIYWVI¹⁶, or ⁶GYGYVQIYWVV¹⁶, or ⁶GYGYLQIYWVL¹⁶ and ⁶⁷GYPPL⁷¹, or ⁶GYGYIQIYWVI¹⁶ and ⁶⁷GYPPI⁷¹, or ⁶GYGYVQIYWVV¹⁶ and ⁶⁷GYPPV⁷¹, wherein the amino acid positions correspond to the amino acid positions of the SEQ ID NO: 1.

In an embodiment, the Fas ligand (FasL) variant comprises an amino acid sequence selected from one of SEQ ID NO: 1 to SEQ ID NO: 6.

In an embodiment, the FasL variant comprises modifications which facilitate transportation of the FasL variant to secretory lysosomes, when expressed in a cell. In an embodiment, the FasL variant comprises modifications which reduce transportation of the FasL variant to cell membrane, when expressed in a cell, and when compared to a FasL without said modifications (i.e., unmodified FasL). In an embodiment, the modifications of the FasL variant comprises at least two, at least three, or at least four, or at least five, or at least six, or at least seven amino acid substitutions when compared to a wild type FasL. In an embodiment, the Fas ligand (FasL) variant is obtained by introducing at least three or four amino acid substitutions to a wild type FasL, In an embodiment, FasL variant can also comprise at least two or three further modification in addition to the at least four amino acid substitutions, wherein the at least one further modification is selected from the group consisting of amino acid substitution, deletions, and insertion. In an embodiment, the mutation(s) introduced to the FasL variant are within the intracellular domain of the FasL variant. In some embodiments, only some of the mutation(s) introduced to the FasL variant are within the intracellular domain of the FasL variant, whereas other(s) may be located in other domain(s) of the FasL variant. In an embodiment, the FasL variant comprises at least one amino acid substitution when compared to a wild type FasL, and wherein the amino acid substitution is configured to facilitate transportation of the FasL variant to secretory lysosomes, when expressed in a cell. In an embodiment, the FasL variant comprises at least two, at least three or at least four amino acid substitutions when compared to a wild type FasL, and wherein the amino acid substitutions are configured to facilitate transportation of the FasL variant to secretory lysosomes, when expressed in a cell. In an embodiment, nucleic acid sequences of the FasL variant of the present disclosure are codon-optimized for expression in mammalian cells, preferably for expression in human cells. According to some embodiments of the invention, substituted amino acids leucine, isoleucine or valine (L; I; V) in the modified forms of the Fas-ligand, such as FasLmod1-6, are encoded by codons that is optimized for the use of human codon for expression in human cells. Unless otherwise specified, a particular nucleic acid sequence of the modified forms of the Fas-ligand implicitly encompasses its conservatively modified variants (eg, degenerate codon substitutions). According to a second example aspect is provided a recombinant cell comprising genetic elements that allow producing the at least one FasL variant of the first aspect. In an embodiment, the recombinant cell is an immunoresponsive cell. In an embodiment, the recombinant cell originates from the lymphoid lineage or from the myeloid lineage. In an embodiment, the recombinant cell is selected from the group consisting of a T cell, a Natural Killer (NK) cell, a B cell, a monocyte and a macrophage. In an embodiment, the T cell is a cytotoxic T lymphocyte (CTL), a regulatory T cell, or a Natural Killer T (NKT) cell. In a preferable embodiment, the recombinant cell is a recombinant NK cell.

In an embodiment, the recombinant cell is an engineered immune cell, such as Natural Killer (NK) cell. In an embodiment, the recombinant cell is a cell of mammalian origin, most preferably a cell of human origin. In an embodiment, the recombinant cell is an engineered immune cell, preferably human immune cell. In an embodiment, the recombinant cell is engineered to express the FasL variant, which FasL variant has an enhanced transportation to secretory lysosomes and reduced transportation to the cell membrane, when compared to a wild type FasL. In an embodiment, the recombinant cells are beneficial, as they have an increased cytotoxicity and survival compared to cells producing unmodified (wild type) FasL. In an embodiment, the increased survival of the recombinant cell is implemented by increased cell viability of the recombinant cells expressing the FasL variants, when compared to corresponding cells expressing unmodified FasL. In an embodiment, the recombinant cell has at least 100%, at least 200% increased cell viability measured after 11 days of culture, when compared to corresponding cells expressing unmodified FasL In an embodiment, the increased cytotoxicity of the recombinant cell is implemented by decreased target (cancerous) cell viability in the presence of the recombinant cells expressing the FasL variants, when compared to corresponding cells expressing unmodified FasL. In an embodiment, the recombinant cell is a human Natural Killer (NK) cell.

In an embodiment, the recombinant cells are derived from umbilical cord blood, peripheral blood, bone marrow, tissue wherein the cells have been infiltrated into said tissue, and/or the recombinant cells are CD34+ cells, iPSCs (induced pluripotent stem cells), ESC (embryonic stem cells) or cells of human NK cell line. It is an object of the present invention to provide a method of transfection of NK cells, wherein cells with the modified forms of the Fas-ligand are individually characterized by higher cytotoxicity compared to unmodified NK cells and greater viability compared to cells producing unmodified FasL, and use of those cells for therapy of cancer and other diseases.

In an embodiment, the recombinant cell is a recombinant NK cell comprising the Fas-ligand variant with the selected amino acid substitutions in intracellular domain that redistribute FasL variant transport towards the secretory lysosomes, preferentially the FasL variant has the amino acid sequence selected from SEQ ID NO: 1 to SEQ ID NO: 6.

According to a third example aspect is provided a vector comprising a polynucleotide encoding the FasL variant of the first aspect and a FasL promoter.

In an embodiment, the vector is an expression vector for the FasL variant. In an embodiment, the vector is an expression vector. In an embodiment, the vector comprises a plasmid expression vector such as a eukaryotic expression vector, a viral vector or an mRNA molecule used as an expression vector. In an embodiment, the expression vector is an mRNA molecule used as an expression vector, such as an mRNA molecule comprising a 5′ cap analogue, a 5′ untranslated region, an open reading frame encoding a Fas-ligand variant encoding protein, a 3′ untranslated region, and a poly(A)-tail. In an embodiment, the expression vector comprises a polynucleotide encoding the FasL variant of the first aspect. In an embodiment, any vector capable of transcription and translation of the FasL variant in a host cell can be used. Any viral vector capable of accepting the coding sequences for the FasL variant molecule(s) to be expressed can be used, for example vectors derived from adenovirus, adeno-associated virus, retrovirus (e.g, lentivirus, Rhabdovirus, murine leukemia virus), or herpes virus. The tropism of the viral vectors can be modified by pseudotyping the vectors with envelope proteins or other surface antigens from other viruses, or by substituting different viral capsid proteins, as appropriate. Such vectors are known to the person skilled in the art. Thus, in an embodiment, the vector is a pseudotyped viral vector. In an embodiment, the expression vector is a plasmid, or any other vector that comprises a nucleic acid sequence that encodes the FasL variant.

In an embodiment, the FasL promoter is a truncated FasL promoter, preferably the truncated promoter has a nucleotide sequence of SEQ ID NO: 13. In an embodiment, the vector comprises a truncated promoter when compared to the wild type FasL promoter.

In an embodiment, the vector comprises a polynucleotide encoding the FasL variant according to any one of SEQ ID NO: 1 to SEQ ID NO: 6. In an embodiment, the vector comprises a polynucleotide according to any one of SEQ ID NO: 7 to SEQ ID NO: 12.

In some embodiments, truncated versions of the FASLG gene promoter may be included in an expression vector encoding modified forms of FasL to enhance the Fas-ligand expression inside the cells. The truncated variant of the FASLG gene promoter (hfasIg) was chosen according with the data of Holtz-Heppelmann and others (Holtz-Heppelmann et al., J Biol Chem. 1998 Feb. 20; 273(8):4416-23; Rivera et al., J Biol Chem. 1998 Aug. 28; 273(35):22382-8; McClure et al., J Biol Chem. 1999 Mar. 19; 274(12):7756-62; Bodor et al., Eur J Immunol. 2002 January; 32(1):203-12). The activity of the truncated promoters is higher than that of the natural one, but lower than that of the widely used CMV promoter. The use of such a promoter makes it possible to achieve a high level of Fas-ligand expression and has a less toxic effect on cells than CMV.

According to another example aspect is disclosed a method of obtaining the recombinant cell of the second aspect, the method comprising: introducing the vector comprising a polynucleotide encoding the FasL variant into a selected cell, thereby providing the recombinant cell. In an embodiment, the method of obtaining the recombinant cell comprises extracting in vivo cells from a mammal, such as human, and preparing the cells by introducing the vector comprising a polynucleotide encoding the FasL variant into the selected cells in vitro. In an embodiment, the recombinant cells expressing FasL variants are cultured, expanded, activated and/or stimulated in vitro. In an embodiment, the method of obtaining the recombinant cell comprises cultivating the recombinant cell in conditions allowing production of the FasL variant polypeptide.

In an embodiment, the method of obtaining the recombinant cell comprises culturing the recombinant cells in the presence of all-trans-retinoic acid (ATRA) in vitro. In some embodiments, addition of ATRA during in vitro cultivation of the recombinant cell leads to the suppression of FasL expression during cell production in vitro, thereby increasing the viability and yield of the recombinant cells. In some embodiments, addition of ATRA during in vitro cultivation of the recombinant cell is configured to provide recombinant cells having increased cytotoxicity. In some embodiments, the method of obtaining the recombinant cell comprises addition of at least 1 μM ATRA during in vitro cultivation of the recombinant cell, which is configured to increase the cell viability and yield. In some embodiments, the method of obtaining the recombinant cell comprises addition of at least 1 μM ATRA for a duration of at least 7 days during in vitro cultivation of the recombinant cell, which is configured to increase the cell viability and yield. Removal of ATRA after the cell cultivation restores the expression level of FasL and the cytotoxic activity of cells, the cells thereby being ready to be used in vivo.

In an embodiment, the method of obtaining the recombinant cell comprises culturing the recombinant cells in the presence of vitamin E or its derivatives in vitro. In some embodiments, addition of vitamin E during in vitro cultivation of the recombinant cell leads to the suppression of FasL expression during cell production in vitro, thereby increasing the viability and yield of the recombinant cells. Removal of vitamin E after the cell cultivation restores the expression level of FasL and the cytotoxic activity of cells, the cells thereby being ready to be used in vivo. In some embodiments, the method of obtaining the recombinant cell comprises addition of at least 1 μM ATRA during in vitro cultivation of the recombinant cell, which is configured to increase the recombinant cell viability and yield. In some embodiments, the method of obtaining the recombinant cell comprises addition of at least 1 μM ATRA for a duration of at least 13 days during in vitro cultivation of the recombinant cell, which is configured to increase the recombinant cell viability and yield. In an embodiment, the method of obtaining the recombinant cell comprises culturing the recombinant cells in the presence of ATRA and vitamin E.

In some embodiments, the recombinant cell comprising the genetic elements that allow producing at least one FasL variant is used in an in vitro application, such as in an in vitro diagnostic application, for example as a reference cell for cell viability or cytotoxicity.

According to some embodiments of the invention, an additional positive effect in obtaining modified cells can be achieved by adding all-trans-retinoic acid (ATRA) when culturing modified NK cells in vitro. ATRA downregulates FasL expression according to data of Yang and others (Yang et al., J Exp Med. 1995 May 1; 181(5):1673-82; Bissonnette et al., Mol Cell Biol. 1995 October; 15(10):5576-85; Cui et al., Cell Immunol. 1996 Feb. 1; 167(2):276-84; Lee et al., Eur J Biochem. 2002 February; 269(4):1162-70).

According to some embodiments of the invention, an additional positive effect in obtaining modified cells can be achieved by adding vitamin E and its derivatives when culturing modified NK cells in vitro. Vitamin E downregulates FasL expression according to data of Li-Weber et al. (J Clin Invest. 2002 September; 110(5):681-90; Lee et al., Nutrients. 2018 Nov. 1; 10(11):1614).

According to some embodiments of the invention, the compositions with the use of a truncated promoter and the addition of ATRA during cultivation leads to the suppression of FasL expression during cell production in vitro. As a result, the viability of the modified NK cells and the yield of the cell product at the end of the cycle of cultivation are increased. After removal of ATRA at the final stage of cultivation, the expression level of FasL and the cytotoxic activity of cells are restored. Thus, the use of a truncated promoter for the expression of modified FasL forms in combination with ATRA makes it possible to obtain a greater yield of NK cells with high cytotoxic activity during in vitro cultivation. In an embodiment, the method of obtaining the recombinant cell comprises:
introducing the vector comprising a polynucleotide encoding the FasL variant into a selected host cell, wherein the vector comprises a truncated FasL promoter, thereby obtaining a recombinant cell,
cultivating the recombinant cells in vitro in the presence of all-trans-retinoic acid (ATRA);
removing ATRA from the cultivated recombinant cells, thereby restoring the expression level of FasL variant and the cytotoxic activity of the recombinant cell.

According to some embodiments of the invention, the compositions with the use of a truncated promoter and the addition of vitamin E or its derivatives during cultivation leads to the suppression of FasL expression during cell production in vitro. As a result, the viability of the modified NK cells and the yield of the cell product at the end of the cycle of cultivation are increased. After removal of vitamin E or its derivatives at the final stage of cultivation, the expression level of FasL and the cytotoxic activity of cells are restored. Thus, the use of a truncated promoter for the expression of modified FasL forms in combination with vitamin E or its derivatives makes it possible to obtain a greater yield of NK cells with high cytotoxic activity during in vitro cultivation.

In an embodiment, the method of obtaining the recombinant cell comprises:
introducing the vector comprising a polynucleotide encoding the FasL variant into a selected host cell, wherein the vector comprises a truncated FasL promoter,
cultivating the recombinant cells in vitro in the presence of vitamin E or its derivatives;
removing vitamin E or its derivatives from the cultivated recombinant cells, thereby restoring the expression level of FasL variant and the cytotoxic activity of the recombinant cell.

According to some embodiments of the invention, an exemplary strategy for improving NK cells for immunotherapy by redistributing FasL transport towards the intracellular depot including to the secretory lysosomes preferentially, enhancing anti-cancer cytotoxicity and improving NK cell survival is shown in FIG. 12.

According to a fourth example aspect, is disclosed a pharmaceutical composition comprising the recombinant cell comprising the FasL variant, and at least one further component selected from a pharmaceutically acceptable excipient, carrier, and/or adjuvant. In an embodiment, the pharmaceutical composition comprises pharmaceutically acceptable excipient. Excipients are inactive substances that serve as vehicles or bulking agents in the pharmaceutical composition. In an embodiment, the pharmaceutically acceptable excipient is selected from one or more of lubricants, preservative, diluent, binders, coating agents, coloring agents, wetting agents, dispersing agents, emulsifying agents (e.g., methylcellulose), pH buffering agents, gelling or viscosity enhancing additives, preservatives, and flavouring and agents. In an embodiment, the pharmaceutical composition comprises a carrier, such as liposomes, nanoparticles, microspheres, and emulsions. Carriers are substances used to deliver the pharmaceutical composition to the target site in the body. In an embodiment, the pharmaceutical composition comprises an adjuvant, such as chemokines, cytokines, other cells or monoclonal antibodies. Adjuvants are substances added to the pharmaceutical composition, to enhance the immune response to said pharmaceutical composition. In an embodiment, the pharmaceutical composition comprises an additional therapeutic agent, pharmaceutically effective compound, and/or a pharmaceutical composition. In an embodiment, the additional therapeutic agent is selected from one or more of: PD1, PDL1, CTLA4, LAG-3 inhibitors such as nivolumab (OPDIVO®), pembrolizumab (KEYTRUDA®), pidilizumab (CureTech), atezolizumab (Tecentriq®), avelumab (Bavencio®), cemiplimab (Libtayo®), dostarlimab (Jemperli), durvalumab (Imfinzi™), Ipilimumab (Yervoy®), and Relatlimab (BMS). According to a fifth example aspect, is disclosed Natural killer (NK) cells modified to express the Fas ligand variants of the first aspect for use in treating a medical condition in an individual in a need thereof, comprising administering to the individual therapeutically effective amount of the NK cells modified to express the Fas ligand variants, wherein the NK cells are configured to have an increased cytotoxicity and survival compared to cells producing unmodified FasL, thereby treating the medical condition. In an embodiment, the use in treatment of a medical condition is use in immunotherapy. In an embodiment, the use in treatment of a medical condition is use in cancer therapy. In an embodiment, the NK cells modified to express the Fas ligand variants in treating a medical condition in an individual in a need thereof, comprises administering to the individual therapeutically effective amount of the pharmaceutical composition of the fourth aspect.

In an embodiment, the therapeutically effective amount depends on the treated disease and the individual, but generally, the therapeutically effective amount can be considered to correspond to an amount wherein a therapeutic effect is obtained. The therapeutic effect in this context can refer to a range from reduction of symptoms of a disease to curing a condition.

In an embodiment, the individual is a mammal, preferably the individual is a human. In an embodiment, the NK cells modified to express the Fas ligand variants are configured to express one or more Fas ligand variants. In an embodiment, the NK cells modified to express the Fas ligand variants are configured to express one or more of the FasLmod1-FasLmod6. In an embodiment, the NK cells modified to express the Fas ligand variants are configured to express a heterogenous population of plurality of FasL variants. In an embodiment, the NK cells modified to express the Fas ligand variants are configured to express only one FasL variant.

In an embodiment, a use of the FasL variant or the recombinant cell, or the pharmaceutical composition according to present disclosure is disclosed, in treating a medical condition in an individual in a need thereof. In an embodiment, a use of the FasL variant or the recombinant cell, or the pharmaceutical composition according to present disclosure is disclosed, in therapeutic treatment of an individual in vivo.

In an embodiment, the NK cells, which are modified to express the Fas ligand variants for use in treating a medical condition, are autologous or allogeneic with respect to the individual. In an embodiment, the medical condition is cancer. In an embodiment, the medical condition is pathogenic infection, such as viral, bacterial, or fungal infection.

In an embodiment, the NK cells, which are modified to express the Fas ligand variants for use in treating a medical condition, are derived from umbilical cord blood, peripheral blood, bone marrow, cells infiltrated into tissues, CD34+ cells, iPSCs (induced pluripotent stem cells), ESC (embryonic stem cells), or cells of a human NK cell line.

In an embodiment, the NK cells, which are modified to express the Fas ligand variants for use in treating a medical condition, are cultured, expanded, activated or stimulated prior to administration to the individual. In an embodiment, the NK cells, which are modified to express the Fas ligand variants for use in treating a medical condition, are cultured, expanded, activated or stimulated in vitro, prior to administration to the individual.

In an embodiment, the NK cells, which are modified to express the Fas ligand variants for use in treating a medical condition, are cultured, expanded, activated or stimulated with various concentrations of all-trans-retinoic acid (ATRA) or with vitamin E or its derivatives, prior to administration to the individual.

In an embodiment, the recombinant cell is a natural killer (NK) cell modified to express Fas ligand variants for use in treating a medical condition in an individual in need thereof, comprising administering to the individual therapeutically effective amount of the natural killer (NK) cells modified to express the Fas ligand variants,

wherein the FasL variants comprise an amino acid sequence selected from SEQ ID NO: 1 to SEQ ID NO: 6, and wherein the modifications comprised by the FasL variants facilitate their transportation to secretory lysosomes of the recombinant cell and increase their cytotoxicity and survival compared to cells producing unmodified FasL, thereby treating a medical condition. In an embodiment, is disclosed a method of treating a medical condition in a patient comprising administering the recombinant cell to the patient. In an embodiment, is disclosed a method for increasing the recombinant cell viability in a therapeutic regimen, the method comprising administering the recombinant cells of the present disclosure to a patient. In an embodiment, is disclosed a method of treating any kind of cancer, including hematological malignancies or solid tumors in an individual in need thereof, comprising administering to the individual therapeutically effective amount of natural killer (NK) cells modified to express the Fas ligand variants, wherein the FasL variants contain amino acid substitutions that facilitate their transportation to secretory lysosomes and increase cytotoxicity and survival compared to cells producing unmodified FasL, thereby treating the medical condition of cancer.

In an embodiment, the method of treating any kind of cancer comprises the NK cells are autologous or allogeneic with respect to the individual.

In an embodiment, the method of treating any kind of cancer comprises the NK cells are derived from umbilical cord blood, peripheral blood, bone marrow, infiltrated into tissues cells, CD34+ cells, iPSCs, ESC or human NK cell lines.

In an embodiment, the method of treating any kind of cancer comprises the NK cells comprise Fas-ligand with the selected amino acid substitutions in intracellular domain that redistribute FasL transport towards the intracellular depot preferentially, wherein the muteins of FasL are encoded by a amino acid sequence selected from SEQ ID NO: 1 to SEQ ID NO: 6.

In an embodiment, the method of treating any kind of cancer comprises said the truncated FasL promoter sequence is SEQ ID NO: 13.

In an embodiment, the method of treating any kind of cancer comprises said NK cells expressing FasL muteins are cultured, expanded, activated or stimulated prior to administration to the individual.

In an embodiment, the method of treating any kind of cancer comprises said NK cells expressing FasL muteins under a control of the truncated FasL promoter are cultured, expanded, activated or stimulated prior to administration to the individual.

In an embodiment, the method of treating any kind of cancer comprises said NK cells expressing FasL muteins are cultured, expanded, activated or stimulated with various concentrations of all-trans-retinoic acid (ATRA) prior to administration to the individual.

In an embodiment, the method of treating any kind of cancer comprises said NK cells expressing FasL muteins are cultured, expanded, activated or stimulated with various concentrations of vitamin E or its derivatives prior to administration to the individual. Unless otherwise stated, all technical and scientific terms used herein have the same meaning as commonly understandable to a person skilled in the art, to which the invention pertains. The materials, methods, and examples described herein are illustrative only and are not intended to be necessarily limiting.

Different non-binding example aspects and embodiments of the present invention have been illustrated in the foregoing. The embodiments in the foregoing are used merely to explain selected aspects or steps that may be utilized in implementations of the present invention. Some embodiments may be presented only with reference to certain example aspects of the invention. It should be appreciated that corresponding embodiments may apply to other example aspects as well.

### SEQUENCE LISTING

SEQ ID NO. 1: Amino acid sequence of the FasL variant FasLmod1, comprising the amino acid substitutions N6G, P8G, P10L, D16L.
SEQ ID NO: 2: Amino acid sequence of the FasL variant FasLmod2, comprising the amino acid substitutions N6G, P8G, P10I, D16I.
SEQ ID NO: 3: Amino acid sequence of the FasL variant FasLmod3, comprising the amino acid substitutions N6G, P8G, P10V, D16V.
SEQ ID NO: 4: Amino acid sequence of the FasL variant FasLmod4, comprising the amino acid substitutions N6G, P8G, P10L, D16L, P67G, L68Y.
SEQ ID NO: 5: Amino acid sequence of the FasL variant FasLmod5, comprising the amino acid substitutions N6G, P8G, P10I, D16I, P67G, L68Y, L71I.
SEQ ID NO: 6: Amino acid sequence of the FasL variant FasLmod6, comprising the amino acid substitutions N6G, P8G, P10V, D16V, P67G, L68Y, L71V.
SEQ ID NO: 7: Nucleotide sequence encoding the FasL variant FasLmod1.
SEQ ID NO: 8: Nucleotide sequence encoding the FasL variant FasLmod2.
SEQ ID NO: 9: Nucleotide sequence encoding the FasL variant FasLmod3.
SEQ ID NO: 10: Nucleotide sequence encoding the FasL variant FasLmod4.
SEQ ID NO: 11: Nucleotide sequence encoding the FasL variant FasLmod5.
SEQ ID NO: 12: Nucleotide sequence encoding the FasL variant FasLmod6.
SEQ ID NO: 13: An oligonucleotide sequence encoding a truncated FasL promoter sequence.
SEQ ID NO: 14: A forward primer oligonucleotide sequence for encoding a truncated FasL gene promoter sequence of SEQ ID NO:13.
SEQ ID NO: 15: A reverse primer oligonucleotide sequence for encoding a truncated FasL gene promoter sequence of SEQ ID NO:13.
SEQ ID NO: 16: An oligonucleotide sequence F1GL.
SEQ ID NO: 17: An oligonucleotide sequence F1GI.
SEQ ID NO: 18: An oligonucleotide sequence F1GV.
SEQ ID NO: 19: An oligonucleotide sequence F2L.
SEQ ID NO: 20: An oligonucleotide sequence F2I.
SEQ ID NO: 21: An oligonucleotide sequence F2V.
SEQ ID NO: 22: An oligonucleotide sequence F3.
SEQ ID NO: 23: An oligonucleotide sequence F4.
SEQ ID NO: 24: An oligonucleotide sequence F5.
SEQ ID NO: 25: An oligonucleotide sequence F6.
SEQ ID NO: 26: An oligonucleotide sequence F6GYL.
SEQ ID NO: 27: An oligonucleotide sequence F6GYI.
SEQ ID NO: 28: An oligonucleotide sequence F6GYV.
SEQ ID NO: 29: An oligonucleotide sequence R1.
SEQ ID NO: 30: An oligonucleotide sequence R2.
SEQ ID NO: 31: An oligonucleotide sequence R2GYL.
SEQ ID NO: 32: An oligonucleotide sequence R2GYI.
SEQ ID NO: 33: An oligonucleotide sequence R2GYV.
SEQ ID NO: 34: An oligonucleotide sequence R3.
SEQ ID NO: 35: An oligonucleotide sequence R4.
SEQ ID NO: 36: An oligonucleotide sequence R5.
SEQ ID NO: 37: An oligonucleotide sequence R6GL.
SEQ ID NO: 38: An oligonucleotide sequence R6GI.
SEQ ID NO: 39: An oligonucleotide sequence R6GV.
SEQ ID NO: 40: An oligonucleotide sequence LNKF1.
SEQ ID NO: 41: An oligonucleotide sequence LNKR1.
SEQ ID NO: 42: An oligonucleotide sequence LNKF3.
SEQ ID NO: 43: An oligonucleotide sequence FasIR_Xho.
SEQ ID NO: 44: An oligonucleotide sequence FasIP_Mlu.
SEQ ID NO: 45: An oligonucleotide sequence ExF.
SEQ ID NO: 46: An oligonucleotide sequence ExR.
SEQ ID NO: 47. An amino acid sequence of the wild type human Fas Ligand.

### LIST OF THE FIGURES

Some example embodiments of the invention will be described with reference to the accompanying drawings, in which:

FIG. 1 depicts a map of the plasmid vector pFasLmod illustrating the point of insertion of certain constructs according to several embodiments into plasmids. Human fasIg promoter—233-685 bp, FasLmod1-6—686-1531 bp, BGH pA—1575-1799 bp, f1 ori—1845-2273 bp, SV40 early promoter—2278-2647 bp, Neo(R)—2683-3477 bp, SV40 pA—3651-3781 bp, pUC origin—4164-4834 bp, Amp(R)—4979-5839 bp (a complementary chain), bIa promoter—5840-5938 bp (a complementary chain). FasLmod position in the pFasLmod vector represents the position wherein the FasL variant (FasLmod1-6) can be inserted.

FIG. 2 is a photomicrograph illustrating the cytotoxicity of NK cells modified with various constructs (vectors) according to several embodiments against target cells HEK293 (cells of a human embryonal kidney). NK cells, NK-FasLmod1, NK-FasLmod2, NK-FasLmod3, NK-FasLmod4, NK-FasLmod5, NK-FasLmod6, were incubated with HEK293 target cells at a ratio of 3:1 (effector:target) for 5 hours and photographed.

FIG. 3 is a photomicrograph illustrating the cytotoxicity of NK cells modified with various constructs according to several embodiments against target cells HeLa (human cervical adenocarcinoma cells). NK cells, NK-FasLmod1, NK-FasLmod2, NK-FasLmod3, NK-FasLmod4, NK-FasLmod5, NK-FasLmod6, were incubated with HeLa target cells at a ratio of 3:1 (effector:target) for 5 hours and photographed.

FIG. 4 is a photomicrograph illustrating the cytotoxicity of NK cells modified with various constructs according to several embodiments against target cells A172 (human glioblastoma cells). NK cells, NK-FasLmod1, NK-FasLmod2, NK-FasLmod3, NK-FasLmod4, NK-FasLmod5, NK-FasLmod6, were incubated with A172 target cells at a ratio of 3:1 (effector:target) for 5 hours and photographed.

FIG. 5 depicts percentage of surviving HeLa target cells after incubation with NK92, NK92-FasLmod1, NK92-FasLmod2, NK92-FasLmod3, NK92-FasLmod4, NK92-FasLmod5, NK92-FasLmod6 cells for 5 hours at a ratio of 2:1 or 5:1 (effector:target).

FIG. 6 depicts percentage of surviving HEK293 target cells after incubation with NK92, NK92-FasLmod1, NK92-FasLmod2, NK92-FasLmod3, NK92-FasLmod4, NK92-FasLmod5, NK92-FasLmod6 cells for 5 hours at a ratio of 2:1 or 5:1 (effector:target).

FIG. 7 depicts percentage of surviving A172 target cells after incubation with NK92, NK92-FasLmod1, NK92-FasLmod2, NK92-FasLmod3, NK92-FasLmod4, NK92-FasLmod5, NK92-FasLmod6 cells for 5 hours at a ratio of 2:1 or 5:1 (effector:target).

FIG. 8 depicts comparative growth kinetics of NK92, NK92-FasLmod1, NK92-FasLmod2, NK92-FasLmod3, NK92-FasLmod4, NK92-FasLmod5, NK92-FasLmod6 cell cultures.

FIG. 9A depicts Fas ligand expression in NK92 cells, transfected with a vector encoding FasLmod1 under transcriptional control of the natural fasIg promoter (1), the truncated fasIg promoter (2), or the cytomegalovirus promoter (3). FIG. 9B shows the growth rate of NK92-FasLmod1 cells with the natural fasIg promoter (1), the truncated fasIg promoter (2), or the cytomegalovirus promoter (3). M—marker.

FIG. 10A depicts Fas Ligand expression in NK92 cells (1), NK92-FasLmod1 cells with a truncated fasIg promoter in the presence of ATRA (2), NK92-FasLmod1 cells with a truncated fasIg promoter after removal of ATRA (3). FIG. 10B shows growth rate of NK92-FasLmod1 cells with truncated fasIg promoter with or without ATRA. NK92 cells were used as controls. FIG. 10C depicts a viability of HeLa target cells after incubation with NK92-FasLmod1 cells with truncated fasIg promoter after incubation with or without ATRA for 5 hours in a ratio of 2:1 or 5:1 (effector:target). NK92 cells were used as controls.

FIG. 11A depicts Fas Ligand expression in NK92 cells (1), NK92-FasLmod1 cells with a truncated fasIg promoter in the presence of vitamin E (2), NK92-FasLmod1 cells with a truncated fasIg promoter after removal of vitamin E (3). FIG. 11B shows proliferation rate of NK92-FasLmod1 cells with truncated fasIg promoter with or without vitamin E (vitE). NK92 cells were used as controls. FIG. 11C depicts a viability of HeLa target cells after incubation with NK92-FasLmod1 cells with truncated fasIg promoter after incubation with or without vitamin E, for 5 hours in a ratio of 2:1 or 5:1 (effector:target). NK92 cells were used as controls.

FIG. 12 is an illustration showing an exemplary strategy to improve the recombinant cells, such as the recombinant NK cells for immunotherapy by redistributing FasL variant transport to the intracellular depot including to the secretory lysosomes preferentially, enhancing anti-cancer cytotoxicity of the recombinant cell and improving NK cell survival. The FIG. 12A) represents the distribution of unmodified FasL (F) inside and on the cell membrane of the NK cells. Only few extracellular vesicles comprising unmodified (F) are released by secretory lysosomes from NK cells upon intercellular contact with cancer tissue (C), the cytotoxic effect of NK cell on cancer tissue being very localized. The FIG. 12 B) represents the distribution of the modified FasL (F), i.e. the FasL variant, inside and on the cell membrane of the recombinant NK cells. When compared to the unmodified (F), the (F) variant is distributed more to the secretory lysosomes (rather than to cell membrane). Therefore, the (F) variants which are released by secretory lysosomes of the recombinant cells upon intercellular contact with cancer tissue (C), are able to reach and act upon cancer (C) tissue which is not in direct vicinity of the recombinant cell, or which is not in direct cell-to-cell contact with the recombinant cell.

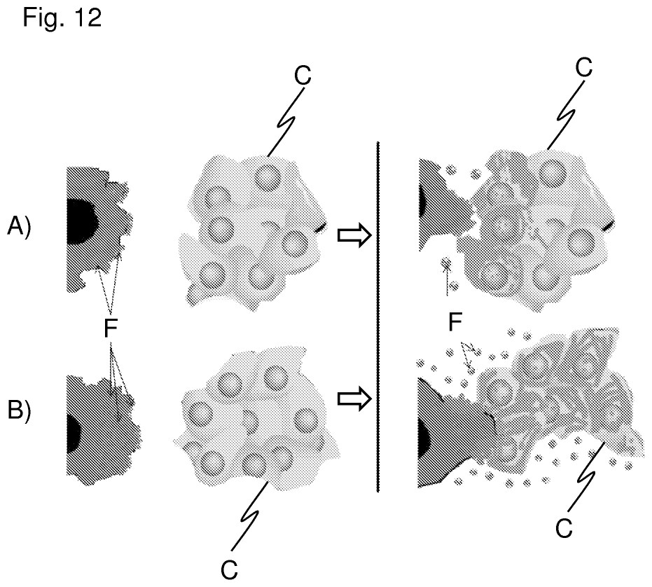

### DETAILED DESCRIPTION OF THE INVENTION

In the following description, like reference signs denote like elements or steps.

### DEFINITIONS

Unless otherwise noted, all technical and scientific terms used herein have a meaning commonly understood by a person skilled in the art. The following references provide the skilled person with a general definition of many of the terms used in the subject matter of the invention disclosed herein: Dictionary of microbiology and molecular biology. (2nd Edition, Singleton, P. & Sainsbury, D. 1988); Concise Dictionary of Biomedicine and Molecular Biology (2nd Edition, 2001, Pei-Show Juo); Oxford Dictionary of Biochemistry and Molecular Biology (2nd Edition, Eds. Richard Cammack et al., 2006); The Dictionary of Cell and Molecular Biology (5th Edition, 2012, Ed. John Lackie).

All publications referred to herein are expressly incorporated by reference into this document for the disclosure and description of the methods and/or materials in connection with which they are cited.

The term “immunotherapy” refers to the treatment of a disease by a method, including the induction, enhancement, suppression or other change in the immune response. Examples of immunotherapy include, but are not limited to, NK cell therapy. It should be understood that the methods disclosed herein enhance the effectiveness of any NK cell therapy.

The term “NK cell” or “natural killer (NK) cell” refers to a subset of peripheral blood lymphocytes defined by the expression of CD56 or CD16 and the absence of the T cell receptor (CD3). In some embodiments the NK cell is derived from umbilical cord blood, peripheral blood, bone marrow, CD34+ cells, iPSCs, ESC or the NK cell is infiltrated into tissues. In some respect the NK cell is human NK cell lines, e. g. NKL (CVCL_0466), YTS (CVCL_D324), NK 3.3 (CVCL_7994), NK-92 (CVCL_2142), KHYG-1 (CVCL_2976), haNK (CVCL_IM23), IaNK (CVCL_VN54) and others. In an embodiment, the NK cell is a modified NK cell, modified to express the Fas ligand variant of present disclosure.

As used herein, the term “Fas ligand” or “FasL” or “CD95L” or “CD178” refers to a type-Il transmembrane protein that belongs to the tumor necrosis factor (TNF) superfamily and induces apoptosis of a cell carrying the death receptor Fas/CD95, or by the reverse signalling pathway. A wild type FasL contains extracellular, transmembrane, and intracellular domains.

As used herein, the term “variant” means a sequence or a fragment of a sequence (nucleotide or amino acid) inserted, substituted or deleted by one or more nucleotides/amino acids, or which is chemically modified, which differs from the corresponding unmodified parent molecule.

The term “FasL variant” or “FasL mutein” or “modified FasL” or “modified form of FasL” means any FasL molecule obtained by site-directed mutagenesis, insertion, substitution, deletion, recombination and/or any other protein engineering method, which leads to FasL variants that differ in their amino acid sequence from the parent FasL, the parent FasL being a wild-type FasL or a FasL variant itself. The terms “wild type FasL”, “wild type”, or “wt” in accordance with the disclosure, describe a FasL with an amino acid sequence found in nature or a fragment thereof. The variant encoding gene can be synthesized, or the parent gene be modified using genetic methods, e.g. by site-directed mutagenesis, a technique in which one or more than one mutations are introduced at one or more defined sites in a polynucleotide encoding the parent polypeptide.

The term “FasL variant” may also be referred to by using the name given to variant, e.g. FasLmod1-6, or a variant according to one of the SEQ ID NO: 1 to SEQ ID NO: 6.

As used herein the “intracellular domain” of the FasL variant refers to the amino acids of the FasL variant corresponding to the amino acids 1-80 of the wild type FasL of the SEQ ID NO: 47, making up the intracellular domain of the wt FasL. For example, the intracellular domains of the FasL variants according to the sequences SEQ ID NO: 1 to SEQ ID NO: 6 comprise the amino acids 1-80 of the sequences SEQ ID NO: 1 to SEQ ID NO: 6, respectively.

As used herein, the term “polypeptide” is an amino acid sequence including a plurality of consecutive polymerized amino acid residues. For purpose of this disclosure, polypeptides include more than 20 amino acid residues. The polypeptide may include modified amino acid residues, naturally occurring amino acid residues not encoded by a codon, and non-naturally occurring amino acid residues. As used herein, a “protein” may refer to a peptide or a polypeptide of any size. A protein may be a receptor protein, a transmembrane protein, a membrane protein, a peptide hormone, an enzyme, an antibody, a regulator, or any other protein.

As used herein, “sequence identity” means the percentage of exact matches of amino acid residues between two optimally aligned sequences over the number of positions where there are residues present in both sequences. When one sequence has a residue with no corresponding residue in the other sequence, the alignment program allows a gap in the alignment, and that position is not counted in the denominator of the identity calculation. As used herein, “sequence alignment” of the amino acid sequences means, aligning the sequences using the AlignX™ module of VectorNTI™ (Invitrogen Corp., Carlsbad, CA) using the default settings.

As used herein, the term “corresponding positions” or “corresponding amino acid position” means aligning at least two amino acid sequences according to identified regions of similarity or identity as pairwise alignment or as multiple sequence alignment, thereby pairing up the corresponding amino acids. An example of corresponding amino acid positions, the sequences SEQ ID NO:1 to 6 are provided, wherein the amino acids 1-281 present in each of said sequences are corresponding amino acids. For example, at the amino acid position 10, the amino acid L of SEQ ID NO: 1 (L¹⁰) and the amino acid I of SEQ ID NO: 2 (I¹⁰) are corresponding amino acids.

As used herein, the superscript numbering used in context of amino acid sequences refers to the amino acid position of the first consequent or the last subsequent amino acid following or preceding the number, respectively. For example with the expression ⁶GYGYL¹⁰ is meant an amino acid sequence, wherein the first listed amino acid G is at the position 6 (G⁶), whereas the last listed amino acid L is at the position 10 (L¹⁰).

As used herein, with the term “Activated natural killer (NK) cells” is meant an activated NK cell, which upon activation, such as contact with a target cell, exhibits enhanced cytotoxic activity, cytokine production (such as interferon-gamma), and/or undergoes proliferation. The term “cytotoxicity” refers to the ability to kill living cells, and specifically describes the characteristics of recombinant cell, such as NK cell activity that kills target cells. The extent of cell death can be expressed as the percentage of target cell death in excess of the background, with total target cell death taken as 100%.

The term “cell survival” refers to the span that encompasses the viability of a cell and its ability to subsist and maintain the integrity of cellular processes. Survival mechanisms ensure that the cell will be able to carry on cellular activities such as metabolism, growth, reproduction, some form of responsiveness, and adaptability.

The term “secretory lysosomes” refers to the lysosome-related effector vesicles (LREVs), which serve as a common storage site for cytotoxic effector proteins and are released only into the immunological synapse formed between the effector and the target cell. As used herein, the term “secretory lysosomes” includes all membrane-bound vesicles that are smaller in diameter than the cell from which they are derived. In the present invention, the term “secretory lysosomes” includes any selected from the group consisting of exosomes, ectosomes and microvesicles, as well as any other vesicles. As used herein, with the term “intracellular depot” is meant any cellular structure or compartment within a cell where certain molecules or substances are stored including also any intracellular secretory lysosomes.

The term “genetic modification” refers to a method of altering the genome of a cell, including, but not limited to, removing a coding or non-coding region or portion thereof, or inserting a coding region or portion thereof. The construct or sequence may include regulatory or control sequences such as start, stop, promoter, signal, secretion, or other sequences used by the cell's genetic machinery. In some embodiments, the target of genetic modification is a cell. In some embodiments, the cell to be modified is an NK cell, which can be obtained from a patient or a donor. The cell may be modified to express an exogenous construct, such as FasL, which is inserted into the cell's genome. In some embodiments, the exogenous construct is a FasL variant, which is inserted into the cell's genome. In some embodiments, the target of genetic modification is FasL.

As used herein, “amino acid substitution” means an amino acid residue replacement with an amino acid residue that is different than the original amino acid in that specific replacement position. The term “amino acid substitution” can refer to both, conservative amino acid substitutions and non-conservative amino acid substitutions, which means the amino acid residue is replaced with an amino acid residue having a similar side chain (conservative), or a different side chain (non-conservative), as the original amino acid residue in that place.

As used herein, “recombinant cell” means any cell type that is genetically modified through transformation, transfection, transduction, or the like with a nucleic acid construct or expression vector comprising a polynucleotide. The term “recombinant cell” encompasses any progeny that is not identical due to mutations that occur during replication. With the terms variant cell, modified cell, engineered cell, or host cells can also be referred to, and used interchangeably with the term “recombinant cell”. The recombinant cell as referred herein refers to the recombinant cell comprising genetic elements that allow producing at least one FasL variant.

The term “promoter” denotes a portion of a gene containing DNA sequences that provide for the binding of RNA polymerase and initiation of transcription. Promoter sequences are commonly but not always found in the 5′ non-coding regions of genes.

As used herein, the term “domain” can be used interchangeably with the term “region” or “site”.

As used herein, the term “trafficking domain” of the intracellular domain of a FasL refers to sequence YXXφ or to sequence GYXXφ, wherein X is any amino acid, and φ is a hydrophobic amino acid selected from leucine, isoleucine or valine (L; I; V).

The following abbreviations are used for amino acids: Alanine (A); Cysteine (C); Aspartic acid (D); Glutamic acid (E); Phenylalanine (F); Glycine (G); Histidine (H); Isoleucine (I); Lysine (K); Leucine (L); Methionine (M); Asparagine (N); Proline (P); Glutamine (Q); Arginine (R); Serine(S); Threonine (T); Valine (V); Tryptophan (W); Tyrosine (Y).

As used herein, the term “comprising” includes the broader meanings of “including”, “containing”, and “comprehending”, as well as the narrower expressions “consisting of” and “consisting only of”.

As used herein, with the amino acid symbol “φ” is meant an amino acid according to the definition of the corresponding context, and for which also a symbol “B” can equally be used. Therefore, wherever the symbol “φ” is used for at least one amino acid, the symbol “B” can also be used instead of the symbol “φ”.

Various aspects of the disclosure of the invention are described in more detail in the following subsections.

### NK Cell Immunotherapy

Immunotherapy, in particular CAR-modified cell therapy, has great potential due to its high cytotoxicity and specificity, and CAR-T-cell immunotherapy is an example of a major form of immunotherapy that is well studied and fairly widely used. However, there are numerous limitations to the use of CAR-modified T cells. The production of personalized CAR-T products is time-consuming and expensive. A drawback of this approach is the necessity to use autologous cells to prevent the induction of a graft vs. host reaction in the patient. CAR-T cells can also cause marked toxic effects by cytokine release syndrome. In addition, the results of CAR-T cell therapy for solid tumors are not optimal. These disadvantages of CAR-T cells have led to great interest in NK-cell therapy. NK cells are cytotoxic lymphocytes of the innate immune system characterized by their ability to spontaneously detect and kill infected or malignant cells and also participate in the regulation of the adaptive immune response by producing a large number of cytokines and chemokines. NK cells use activating receptors to recognize germline-encoded ligands upregulated on cancer cells without requiring tumor neoantigen presentation by MHC molecules as T cells do. Activated NKs are capable of destroying tumor cells by a) releasing cytoplasmic granules containing perforin and granzyme; b) expressing and secreting TNF family proteins such as FasL and TRAIL that induce tumor cell apoptosis, and c) antibody-dependent cellular cytotoxicity mediated by Fc-receptor CD16. Overexpression of Fas ligand on the plasma membrane (in lipid rafts) can lead to the death of the cells producing it (AICD). Retention of Fas ligand inside the cell, in particular in secretory lysosomes, is both a protective mechanism and one of the key factors of cytotoxic activity of NK cells. The ability to regulate AICD in NK cells is essential for NK cell therapy of cancer. Accordingly, in several embodiments of the present invention there is provided a method to solve this problem by creating genetic constructions that confer both greater NK recombinant cell survival and increased cytotoxicity of the natural killer cells in order to promote NK killing of target cells. The genetic constructions are designed to redistribute Fas-ligand transport towards the intracellular depot, but not to the plasma membrane.

### FAS Ligand Intracellular Domain

As mentioned above, NK cells can recognize and destroy tumor cells and infected cells by means of FasL that induces target cell apoptosis. Fas ligand (FasL or CD95L or CD178) is a type-Il transmembrane protein that belongs to the tumor necrosis factor (TNF) family and induces apoptosis through the death receptor Fas/CD95, or by the reverse signalling pathway. FasL contains extracellular, transmembrane, and intracellular domains. The extracellular part is responsible for recognition of the corresponding receptors, Fas-antigen and DcR3, as well as for ligand self-association (Orlinick et al., J Biol Chem. 1997 Dec. 19; 272(51):32221-9). The transmembrane region of FasL is responsible for the “anchoring” and/or movement of this molecule in/on the plasma membrane. The intracellular part of FasL is required for sorting into secretory lysosomes, translocation of the ligand into rafts, the “signaling platforms” of the plasma membrane, and for FasL-dependent reverse signaling. The 45-65 a.a. polyproline region (PRD), is required for interaction with a number of enzymes and adaptor proteins, as well as directed ligand transport. The ubiquitinylation sites of lysine residues at positions 72 and 73 and the phosphorylation of tyrosine residues (7, 9, and 13 a.s.) play a role in the intracellular distribution of the ligand (Zuccato et al., J Cell Sci. 2007 Jan. 1; 120 (Pt 1):191-9). The target signal directing FasL proteins to the lysosomal compartment includes the amino acid sequence YXXφ, where X is any amino acid, φ is a hydrophobic amino acid (leucine, isoleucine or valine (L; I; V)). Chimeric proteins containing this amino acid motif are efficiently directed to this lysosomal compartment (Wu et al., Proc Natl Acad Sci USA. 1995 Dec. 5; 92(25):11671-5). As noted herein, the ability to reduce AICD in NK cells has important implications to NK cell therapy. In an embodiment, the FasL redistribution from cell surface to lysosomal compartment can reduce AICD of activated NK cells. Accordingly, in one embodiment, the intracellular domain of the Fas-ligand variant comprises the YXXφ site (9YPQI12) responsible for its transport into secretory lysosomes. In some embodiments, glycine residue preceding tyrosine promotes FasL variant protein transport into the lysosomal compartment.

In an embodiment, the presence of several sequentially arranged “trafficking domains” (GYXXφ) enhances protein targeting into the lysosomal compartment. In some embodiments, modified forms of the Fas-ligand comprise amino acid substitutions resulting in several sequentially repeated GYXXφ sites, that promotes their transport to secretory lysosomes.

In certain embodiments, the Fas-ligand variant comprises the amino acid sequence set forth in SEQ ID NO: 1. An exemplary nucleic acid sequence encoding the amino acid sequence of SEQ ID NO: 1 is set forth in SEQ ID NO: 7.

In certain embodiments, the Fas-ligand variant comprises the amino acid sequence set forth in SEQ ID NO: 2. An exemplary nucleic acid sequence encoding amino acids of SEQ ID NO: 2 is set forth in SEQ ID NO: 8.

In certain embodiments, the Fas-ligand variant comprises the amino acid sequence set forth in SEQ ID NO: 3. An exemplary nucleic acid sequence encoding amino acids of SEQ ID NO: 3 is set forth in SEQ ID NO: 9.

In certain embodiments, the Fas-ligand variant comprises the amino acid sequence set forth in SEQ ID NO: 4. An exemplary nucleic acid sequence encoding amino acids of SEQ ID NO: 4 is set forth in SEQ ID NO: 10.

In certain embodiments, the Fas-ligand variant comprises the amino acid sequence set forth in SEQ ID NO: 5. An exemplary nucleic acid sequence encoding amino acids of SEQ ID NO: 5 is set forth in SEQ ID NO: 11.

In certain embodiments, the Fas-ligand variant comprises the amino acid sequence set forth in SEQ ID NO: 6. An exemplary nucleic acid sequence encoding amino acids of SEQ ID NO: 6 is set forth in SEQ ID NO: 12.

Because of the degeneracy of the genetic code, a large number of functionally identical nucleic acids encode any given protein. For instance, the codons CTT, CTC, CTA, CTG, TTA and TTG all encode the amino acid leucine. Thus, at every position where a leucine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. In some embodiments, substituted amino acids (leucine, isoleucine or valine (L; I; V) in the modified forms of the Fas-ligand, FasLmod1-6, are encoded by codons that is optimized for the use of human codon for expression in human cells. Unless otherwise specified, a particular nucleic acid sequence of the modified forms of the Fas-ligand implicitly encompasses its conservatively modified variants (eg, degenerate codon substitutions).

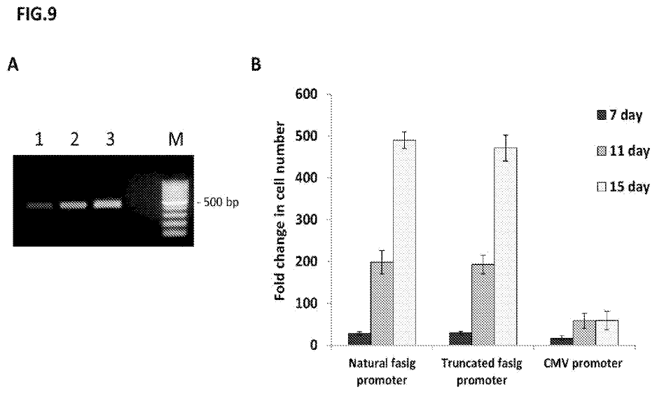

Due to the fact that increased expression of FasL can cause death of the cells producing it, a shortened hfasIg promoter can be included in the expression vector encoding the modified form of FasL to increase the viability of the modified NK cells. The sequence of the shortened vector was chosen based on data from Holtz-Heppelmann et al. where it was shown on the Jurkat T-cell line that deletion of the −2365 −452 region of the FASLG gene promoter leads to a several-fold increase in gene. The activity of the shortened (truncated) promoter is higher than that of the natural promoter, but lower than that of the commonly used viral CMV promoter. The use of such a truncated promoter makes it possible to achieve a high level of Fas ligand expression. At the same time, the truncated promoter has less toxic effect on cells than CMV, thereby increasing the recombinant cell viability, which is illustrated in FIG. 9 B. Accordingly, in some embodiments, truncated version of hfasIg promoter may be included in an expression vector encoding modified forms of FasL to enhance the Fas-ligand expression inside the cells.

The sequence of a truncated version of the FasL gene promoter (hfasIg promoter) is set forth in SEQ ID NO: 13.

An additional positive effect on the viability of modified cells can be achieved by adding all-trans-retinoic acid (ATRA) during the cultivation of modified NK cells in vitro. ATRA is known to suppress FasL expression and, consequently, death of activated thymocytes and T cells (Iwata et al., J Immunol. 1992 Nov. 15; 149(10):3302-8; Szondy et al., J Infect Dis. 1998 November; 178(5):1288-98). One of the mechanisms of ATRA action is based on inhibition of NFAT (nuclear factors of activated T-cells) protein activity (Lee et al., Eur J Biochem. 2002 February; 269(4):1162-70). NFAT is one of the main effectors that trigger FasL transcription by interacting with the GGAAA sequence at position-276 relative to the transcription initiation point. Accordingly, in some embodiments of the invention, an additional positive effect in obtaining modified recombinant cells can be achieved by adding all-trans-retinoic acid (ATRA) when culturing recombinant cells such as modified NK cells in vitro. NK cells can be cultured in the presence of ATRA prior to addition of the recombinant cells to the target cells. In some embodiments of the invention, the compositions with the use of a truncated promoter and the addition of ATRA during cultivation leads to the suppression of FasL expression during cell production in vitro. As a result, the viability of the modified NK cells and the yield of the cell product at the end of the cycle of cultivation are increased. After removal of ATRA at the final stage of cultivation, the expression level of FasL and the cytotoxic activity of cells are restored. Thus, the use of a truncated promoter for the expression of modified FasL forms in combination with ATRA makes it possible to obtain a greater yield of NK cells with high cytotoxic activity during in vitro cultivation.

The natural free radical scavenger vitamin E suppresses the activity of the transcription factors NF-kappa B and AP-1, thus blocking expression of CD95L and preventing AICD of immune cells. Administration of vitamin E suppresses CD95L mRNA expression and protects immune cells from CD95-mediated apoptosis. Accordingly, in some embodiments, an additional positive effect in obtaining modified cells can be achieved by adding vitamin E and its derivatives when culturing modified NK cells in vitro. In some embodiments of the invention, the compositions with the use of a truncated promoter and the addition of vitamin E or its derivatives during cultivation leads to the suppression of FasL expression during cell production in vitro. As a result, the viability of the modified NK cells and the yield of the cell product at the end of the cycle of cultivation are increased. After removal of vitamin E or its derivatives at the final stage of cultivation, the expression level of FasL and the cytotoxic activity of cells are restored. Thus, the use of a truncated promoter for the expression of modified FasL forms in combination with vitamin E or its derivatives makes it possible to obtain a greater yield of NK cells with high cytotoxic activity during in vitro cultivation.

### Compositions and Uses

In some embodiments, the pharmaceutical composition comprising NK cells disclosed herein comprises a pharmaceutically acceptable carrier, diluent, emulsifier, preservative and/or adjuvant. In some embodiments, the composition comprises an excipient. Suitable carrier and their formulation are described, for example, in Remington: The Science and Practice of Pharmacy (23rd Edition, Adejare A., Ed., Academic Press, 2020). The pharmaceutical compositions should not include agents that may inactivate or kill NK cells. In some embodiments, the pharmaceutical composition comprises a physiological solution, preferably a phosphate-buffered saline or sterile physiological solution or tissue culture medium.

The present invention provides methods of treating medical condition such as hematological malignancies in a subject. In certain embodiments, the method comprises administering to the subject an effective amount of pharmaceutical composition or recombinant cells, such as NK cells, wherein the NK cell comprises the modified Fas ligand variants, that increase their cytotoxicity and survival.

The present invention provides methods of treating a solid tumor in a subject. In certain embodiments of the invention, the method comprises administering to a subject an effective amount of NK cells, wherein the NK cells include modified variants of the Fas ligand, thereby increasing their cytotoxicity and survival rate.

The methods disclosed herein may be used to treat cancer in a subject, reduce tumor size, kill tumor cells, prevent tumor growth, prevent tumor recurrence, prevent tumor metastasis, induce remission in a patient, or any combination thereof. In some embodiments, the methods elicit a complete response. In other embodiments, the methods cause a partial response.

The present invention provides methods of preventing and/or treating pathogenic infection in a subject. In certain embodiments of the invention, the method comprises administering to the subject an effective amount of NK cells, where the NK cells include modified variants of the Fas ligand, thereby increasing their cytotoxicity and survival rate. In some embodiments, the pathogen is selected from the group consisting of a virus, bacterium, fungus, parasite, and protozoa capable of causing disease.

A variety of additional therapeutic agents may be used in conjunction with the compositions described herein. For example, potentially useful additional therapeutic agents include, but are not limited to, PD1, PDL1, CTLA4, LAG-3 inhibitors such as nivolumab (OPDIVO®), pembrolizumab (KEYTRUDA®), pidilizumab (CureTech), atezolizumab (Tecentriq®), avelumab (Bavencio®), cemiplimab (Libtayo®), dostarlimab (Jemperli), durvalumab (Imfinzi™), Ipilimumab (Yervoy®), Relatlimab (BMS).

The skilled person can readily determine the amount of cells and optional additives and/or carrier in the compositions and to be administered. For any composition to be administered to an animal or human, the following can be determined: toxicity by determining the lethal dose (LD) and LD50 in a suitable animal model; the dosage of the composition(s), the concentration of components therein and the time of administration of the composition(s) that induce a suitable response.

The following examples are put forth so as to provide those of ordinary skill in the art with a complete disclosure and description of how to make and use the presently disclosed cells and compositions, and are not intended to limit the scope of what the inventors regard as their invention.

### EXAMPLES

### Example 1-Construction of Expression Vectors Encoding Modified Forms of Fas Ligand

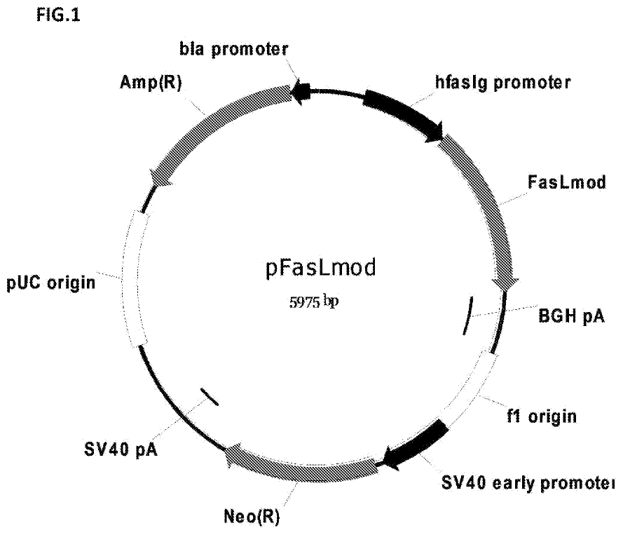

The expression vector with a length of 5975 bp, the physical map of which is shown in FIG. 1, consists of: 233-685 bp.—hfasIg promoter; 686-1531 bp—FasLmod1-6 (in FIG. 1 indicated as FasLmod); 1575-1799 bp—BGH pA; 1845-2273 bp.—f1 ori; 2278-2647 bp—SV40 early promoter; 2683-3477 bp—Neo(R); 3651-3781 bp—SV40 pA; 4164-4834 bp—pUC origin; 4979-5839 bp (complementary chain)—Amp(R); 5840-5938 bp (complementary chain)—bIa promoter.

A fragment representing a shortened FASLG gene promoter (hfasIg promoter) was synthesized by polymerase chain reaction with primers hfasIgprom_f and hfasIgprom_r, SEQ ID NO: 14 and SEQ ID NO: 15, respectively. The primer sequences for the polymerase chain reaction were designed with the OLIGO 4.0 software.

hfaslgprom_f
ttatagccccactgaccattctcctgtagctg
hfaslgprom_r
ctgcatggcagctggtgagtcaggc

Chromosomal DNA from peripheral blood mononuclear cells was used as a matrix. “Thermo Fisher Scientific” Pfu DNA polymerase was used for synthesis according to the manufacturer's instructions. PCR reactions were performed in an Eppendorf thermal cycler. 10 ng of DNA was used as a template in a reaction volume of 25 μl containing 20 mM Tris-HCl (pH 8.8 at 25 °C), 10 mM (NH4) 2SO4, 10 mM KCl, 0.1 mg/mL BSA, 0.1% (v/v) Triton X-100, 2 mM MgSO4, 0.2 mM dNTPs (Thermo Scientific, USA), 0.4 UM of the primers and 1.25 U of PFU DNA polymerase (Thermo Scientific, USA). The amplification protocol was as follow: an initial denaturation at 95 °C for 2 min, followed by 30 cycles at 95 °C for 30 s, 59 °C for 30 s, 72 °C for 1 min, and a final extension at 72 °C for 10 min. The size of each amplification product was resolved by electrophoresis in a 1.2% agarose gel (w/v) prepared in TAE buffer (40 mM Tris, pH 8.3, 20 mM acetic acid, 1 mM EDTA) with 0.4 μg/ml ethidium bromide. 6×DNA Loading Dye containing 10 mM Tris-HCl (pH 7.6) 0.03% bromophenol blue, 0.03% xylene cyanol FF, 60% glycerol, 60 mM EDTA was used for loading samples.

Fragments encoding sequences 1-258 bp of the modified Fas ligand were synthesized by polymerase chain reaction with overlapping oligonucleotides using the method described by Stemmer et al. (Gene. 1995 Oct. 16; 164(1):49-53).

The following oligonucleotide mixture was used to synthesize FasLmod1:

F1GL, F2L, F3, F4, F5, F6, R1, R2, R3, R4,
R5, R6GL.

The following oligonucleotide mixture was used to synthesize FasLmod2:

F1GI, F2I, F3, F4, F5, F6, R1, R2, R3, R4,
R5, R6GI.

The following oligonucleotide mixture was used to synthesize FasLmod3:

F1GV, F2V, F3, F4, F5, F6, R1, R2, R3, R4,
R5, R6GV.

The following oligonucleotide mixture was used to synthesize FasLmod4:

F1GL, F2L, F3, F4, F5, F6GYL, R1, R2GYL, R3,
R4, R5, R6GL.

The following oligonucleotide mixture was used to synthesize FasLmod5:

F1GI, F2I, F3, F4, F5, F6GYI, R1, R2GYI, R3,
R4, R5, R6GI.

The following oligonucleotide mixture was used to synthesize FasLmod6:

F1GV, F2V, F3, F4, F5, F6GYV, R1, R2GYV, R3,
R4, R5, R6GV.

PCR reactions were performed in an Eppendorf thermal cycler. Equal volumes of oligonucleotides were combined to a final concentration 100 μM mixed oligonucleotides. 0.5 μl of oligonucleotide mixture was used as a template in a reaction volume of 25 μl containing 20 mM Tris-HCl (pH 8.8 at 25 °C), 10 mM (NH4)2SO4, 10 mM KCl, 0.1 mg/mL BSA, 0.1% (v/v) Triton X-100, 2 mM MgSO4, 0.2 mM dNTPs (Thermo Scientific, USA), 0.4 UM of the primers and 1.25 U of PFU DNA polymerase (Thermo Scientific, USA). PCR reactions were performed as follow: an initial denaturation at 95 °C for 2 min, followed by 55 cycles at 95 °C for 30 s, 52 °C for 30 s, 72 °C for 30 min, and a final extension at 72 °C for 5 min. “Thermo Fisher Scientific” Pfu DNA polymerase was used for synthesis according to the manufacturer's instructions. The size of each amplification product was resolved by electrophoresis in a 1.2% agarose gel (w/v) prepared in TAE buffer (40 mM Tris, pH 8.3, 20 mM acetic acid, 1 mM EDTA) with 0.4 μg/ml ethidium bromide. 6×DNA Loading Dye containing 10 mM Tris-HCl (pH 7.6) 0.03% bromophenol blue, 0.03% xylene cyanol FF, 60% glycerol, 60 mM EDTA was used for loading samples.

At the next stage, the synthesized fragments were amplified using LNKF1 and LNKR1 primers, SEQ ID NO: 40 and SEQ ID NO: 41, respectively.

8 μl of synthesized fragments mix was used as a template in a reaction volume of 25 μl containing 20 mM Tris-HCl (pH 8.8 at 25 °C), 10 mM (NH4)₂SO₄, 10 mM KCl, 0.1 mg/mL BSA, 0.1% (v/v) Triton X-100, 2 mM MgSO4, 0.2 mM dNTPs (Thermo Scientific, USA), 0.4 μM of the primers and 1.25 U of PFU DNA polymerase (Thermo Scientific, USA). PCR reactions were performed as follow: an initial denaturation at 95 °C for 2 min, followed by 25 cycles at 95 °C for 30 s, 59 °C for 30 s, 72 °C for 30 min, and a final extension at 72 °C for 5 min. “Thermo Fisher Scientific” Pfu DNA polymerase was used for synthesis according to the manufacturer's instructions. The size of each amplification product was resolved by electrophoresis in a 1.2% agarose gel (w/v) prepared in TAE buffer (40 mM Tris, pH 8.3, 20 mM acetic acid, 1 mM EDTA) with 0.4 μg/ml ethidium bromide. 6×DNA Loading Dye containing 10 mM Tris-HCl (pH 7.6) 0.03% bromophenol blue, 0.03% xylene cyanol FF, 60% glycerol, 60 mM EDTA was used for loading samples.

The fragment encoding the sequence 231-846 bp of the Fas ligand was synthesized by polymerase chain reaction with primers LNKF3 and FasIR_Xho, SEQ ID NO: 42 and SEQ ID NO: 43, respectively.

The plasmid vector pcDNA4/TO-FasL previously described by Glukhova et al., 2018 (17), was used as a matrix. 5 ng of plasmid DNA was used as a template in a reaction volume of 25 μl containing 20 mM Tris-HCl (pH 8.8 at 25 °C), 10 mM (NH4)2SO4, 10 mM KCl, 0.1 mg/mL BSA, 0.1% (v/v) Triton X-100, 2 mM MgSO4, 0.2 mM dNTPs (Thermo Scientific, USA), 0.4 μM of the primers and 1.25 U of PFU DNA polymerase (Thermo Scientific, USA). PCR reactions were performed as follow: an initial denaturation at 95 °C for 2 min, followed by 30 cycles at 95 °C for 30 s, 59 °C for 30 s, 72 °C for 80 s, and a final extension at 72 °C for 10 min. “Thermo Fisher Scientific” Pfu DNA polymerase was used for synthesis according to the manufacturer's instructions. The size of each amplification product was resolved by electrophoresis in a 1.2% agarose gel (w/v) prepared in TAE buffer (40 mM Tris, pH 8.3, 20 mM acetic acid, 1 mM EDTA) with 0.4 μg/ml ethidium bromide. 6×DNA Loading Dye containing 10 mM Tris-HCl (pH 7.6) 0.03% bromophenol blue, 0.03% xylene cyanol FF, 60% glycerol, 60 mM EDTA was used for loading samples.

The FasIP_Mlu (SEQ ID NO: 44) and FasIR_Xho (SEQ ID NO: 43) primers were used to synthesize DNA fragments encoding the FasLmod 1 (SEQ ID NO: 1)-FasLmod 6 (SEQ ID NO: 6) sequences and the truncated hfasIg promoter.

A mixture of DNA fragments representing the truncated hfasIg promoter, a fragment encoding the sequence 1-258 bp of FasLmod1-FasLmod6, and a fragment encoding the sequence 231-846 bp of Fas ligand were used as a matrix. 1 ng of a fragment encoding the sequence 1-258 bp of FasLmod1-FasLmod6, and 2 ng of a fragment encoding the sequence 231-846 bp of Fas ligand was used as a template in a reaction volume of 25 μl containing 20 mM Tris-HCl (pH 8.8 at 25 °C), 10 mM (NH4)2SO4, 10 mM KCl, 0.1 mg/mL BSA, 0.1% (v/v) Triton X-100, 2 mM MgSO4, 0.2 mM dNTPs (Thermo Scientific, USA), 0.4 μM of the primers and 1.25 U of PFU DNA polymerase (Thermo Scientific, USA). PCR reactions were performed as follow: an initial denaturation at 95 °C for 2 min, followed by 30 cycles at 95 °C for 30 s, 59 °C for 30 s, 72 °C for 105 s, and a final extension at 72 °C for 10 min. “Thermo Fisher Scientific” Pfu DNA polymerase was used for synthesis according to the manufacturer's instructions. The size of each amplification product was resolved by electrophoresis in a 1.2% agarose gel (w/v) prepared in TAE buffer (40 mM Tris, pH 8.3, 20 mM acetic acid, 1 mM EDTA) with 0.4 μg/ml ethidium bromide. 6×DNA Loading Dye containing 10 mM Tris-HCl (pH 7.6) 0.03% bromophenol blue, 0.03% xylene cyanol FF, 60% glycerol, 60 mM EDTA was used for loading samples.

The fragments encoding the sequences of the modified Fas ligand with the hfasIg promoter were cloned into the pcDNA3.1 (+) vector (Invitrogen) at the MluI and XhoI restriction sites. DNA sequences of the selected plasmid vectors pFasLmod 1, pFasLmod 2, pFasLmod3, pFasLmod4, pFasLmod5, pFasLmod6 were confirmed by DNA sequencing.

Modified NK cells were obtained by introducing plasmid vectors pFasLmod1, pFasLmod2, pFasLmod3, pFasLmod4, pFasLmod5, and pFasLmod6 into cells using Lipofectamine 2000 or Lipofectamine 3000 transfection (Thermo Fisher Scientific) or electroporation using a Gene Pulser Xcell device (Bio-Rad) according to the manufacturer's instructions.

### Example 2—Changes in Target Cell Morphology During Interaction with NK Cells

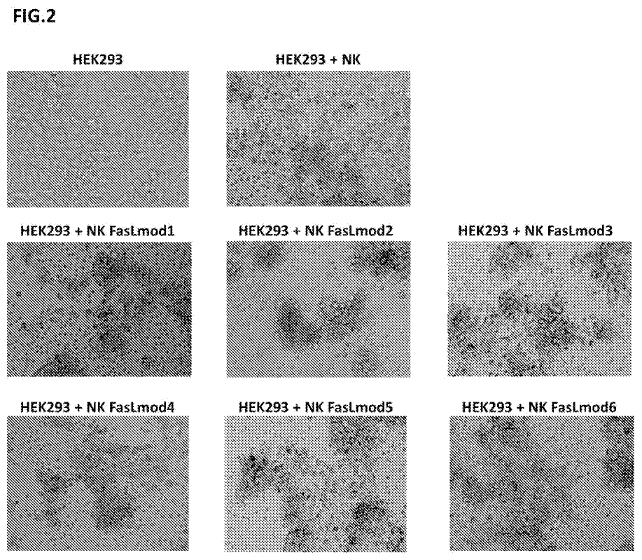

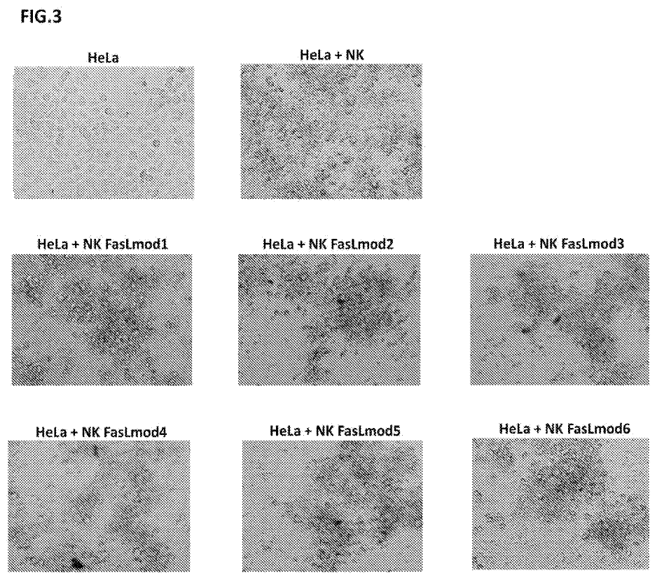

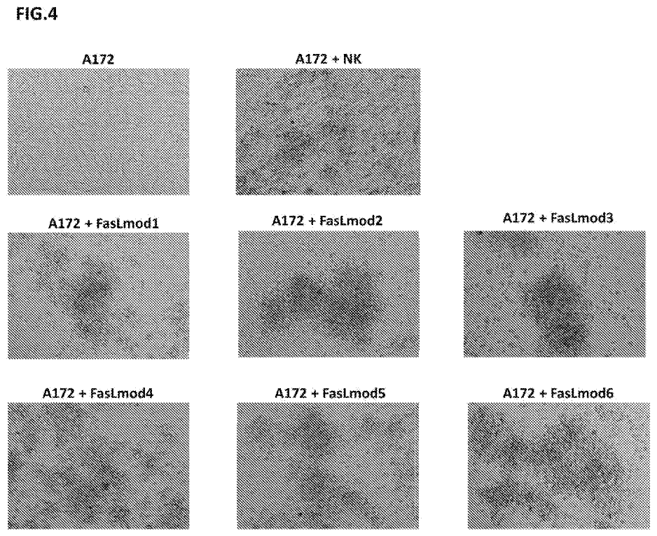

Human NK cells were isolated from peripheral blood mononuclear cell samples from a healthy donor using the NK Cell Isolation Kit (Miltenyi Biotec). NK cells were transfected with the pFasLmod1, pFasLmod2, pFasLmod3, pFasLmod4, pFasLmod5, and pFasLmod6 vectors by electroporation using the Gene Pulser Xcell System (Bio-Rad) according to the manufacturer's instructions. Three days after electroporation, the cells were purified using the Dead Cell Removal Kit (Miltenyi Biotec) and then incubated in DMEM medium containing 500 iu/ml IL-2, 10% FBS, for 6 h with target cells HEK293 (transformed human embryonic kidney cells) (FIG. 2), HeLa (human cervical adenocarcinoma) (FIG. 3), or A172 (human glioblastoma) (FIG. 4) in a 1:3 ratio (target:effector). Cells were observed under phase-contrast optics with a 10× objective.

The data in FIG. 2-4 illustrate typical morphological changes induced in susceptible tumor target cells during an interaction with NK cells. All lines of target cells tested underwent morphological changes associated with cell death. Dying tumor target cells showed rounding-up, shrinkage, plasma membrane blebbing and the presence of apoptotic bodies (Ziegler, Groscurth. News Physiol Sci. 2004 June; 19:124-8). At the same time NK cells completely rounded up and form multi-cellular clusters which surrounded and covered target cells. It is known that these homotypic NK-NK interactions that shape multicellular clusters are critical for optimal cytolytic activation of NK cells, IFN-γ secretion and elimination of tumor cells in vivo (Lee et al., Blood. 2006 Apr. 15; 107(8):3181-8; Kim et al., Sci Rep. 2017 Jan. 11; 7:40623).

All NK cells tested caused HEK293 cell death. However, all modified NK cells were more devastating to target cells compared to control NK cells. Noteworthy is the greater number of live NK-FasLmod cells compared to mock NK cells. The most pronounced changes in the morphology of HEK293 cells were observed when target cells collided with NK-FasLmod2 cells.

All tested NK cells resulted in death of Hela cells (FIG. 3). However, all modified NK cells were more destructive to target cells compared to control NK cells. Also noteworthy is the higher number of live NK-FasLmod cells compared with unmodified NK cells. The most pronounced changes in HeLa cell morphology were observed when target cells encountered with NK-FasLmod2 or NK-FasLmod4 cells.

All NK cells tested resulted in A172 cell death (FIG. 4). However, all modified NK cells were more detrimental to target cells compared to control NK cells. Also of note is the higher number of live NK-FasLmod cells compared with mock NK cells. The most pronounced changes in the morphology of A172 cells were observed when target cells interacted with NK-FasLmod2, NK-FasLmod3, or NK-FasLmod5 cells.

Taken together, these data demonstrate that, in accordance with several embodiments disclosed herein, NK cells that express the FasL muteins are able to be activated and successfully generate enhanced cytotoxic effects against tumor target cells.

### Example 3—Assessment of Cytotoxic Activity of NK92 and NK92-FasLmod1, NK92-FasLmod2, NK92-FasLmod3, NK92-FasLmod4, NK92-FasLmod5, NK92-FasLmod6 Cells Against HeLa Target Cells (Human Cervical Adenocarcinoma)

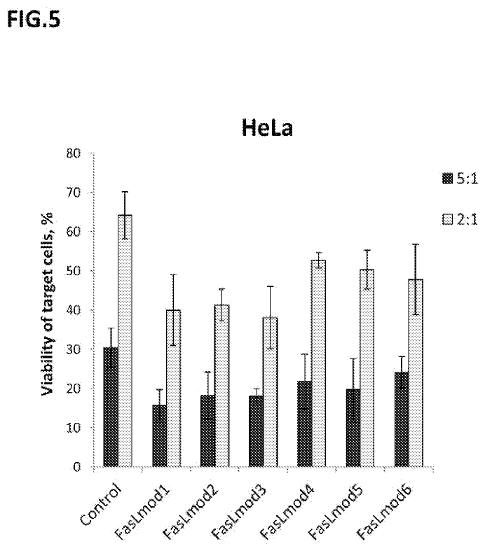

To assess the potency of the modified NK92 cells, cytotoxicity assays were performed using NK cell-sensitive cell lines, HEK293, HeLa, and A172 cells. NK92 cells were transfected with the pFasLmod1, pFasLmod2, pFasLmod3, pFasLmod4, pFasLmod5, and pFasLmod6 vectors by electroporation using the Gene Pulser Xcell System (Bio-Rad) according to the manufacturer's instructions. After electroporation, cells were incubated in αMEM selection medium supplemented with 2 mM L-glutamine, sodium bicarbonate (1.5 g/L), 0.2 mM inositol, 0.1 mM 2-mercaptoethanol, 0.02 mM folic acid, 400 U/ml recombinant interleukin 2, 25% fetal calf serum, 50 μg/ml G418. Target cells (HEK293, HeLa, or A172 cells) were seeded/seeded/planted into the wells of a 96-well plate. The next day, control NK92 cells and modified (NK92-FasLmod1, NK92-FasLmod2, NK92-FasLmod3, NK92-FasLmod4, NK92-FasLmod5, and NK92-FasLmod6) cells were added to the wells of the 96-well plate with the previously seeded target cells and incubated for 5 h at 370 C and 5% CO2 at a ratio of 2:1 or 5:1 (effector:target). The wells were then washed with buffered saline and the number of surviving target cells was estimated by staining with neutral red dye (Wallach J Immunol. 1984 May; 132(5):2464-9). Data summarizing the percent cytotoxicity of different NK92 cell lines against target cells at two E:T ratios are shown in FIGS. 5-7 (all experiments were performed in triplicate).

As depicted in FIG. 5, NK92 cells expressing FasL muteins had a significantly higher cytotoxicity against Hela cells as compared to mock NK92 cells. Even at a low E:T ratio of 2:1, 60%, 57%, 62%, 45%, 50% and 52% of Hela cells were killed by NK92-FasLmod1, NK92-FasLmod2, NK92-FasLmod3, NK92-FasLmod4, NK92-FasLmod5, NK92-FasLmod6 cells, respectively. The mock NK-92 cytotoxicity was 35% at this ratio. At the E:T ratio of 5:1 the cytotoxic effects of NK92-FasL muteins were even more pronounced. NK92-FasLmod1, NK92-FasLmod2, NK92-FasLmod3, NK92-FasLmod4, NK92-FasLmod5, NK92-FasLmod6 cells killed 85%, 83%, 82%, 78%, 80% and 75% of Hela cells, respectively. NK92-FasLmod1, NK92-FasLmod2 and NK92-FasLmod3 cells demonstrated the highest potency of killing Hela cells comparing to other modified NK92 cells.

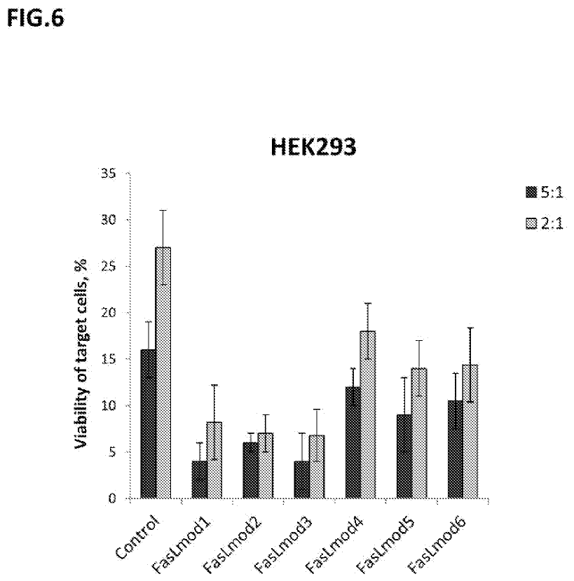

FIG. 6 shows the death rates of HEK293 cells when co-cultured with NK92 cells expressing FasL muteins, in the ratios of 2:1 and 5:1, demonstrating a significant increase in killing compared to mock NK92 cells. At the E:T ratio of 2:1 92%, 93%, 94%, 81%, 87% and 86% of HEK293 cells were killed by NK92-FasLmod1, NK92-FasLmod2, NK92-FasLmod3, NK92-FasLmod4, NK92-FasLmod5, NK92-FasLmod6 cells, respectively. At the E:T ratio of 5:1 NK92-FasLmod1, NK92-FasLmod2, NK92-FasLmod3, NK92-FasLmod4, NK92-FasLmod5, NK92-FasLmod6 cells killed 97%, 94%, 97%, 88%, 91% and 89% of HEK293 cells, respectively. As with Hela cells, NK92-FasLmod1, NK92-FasLmod2, and NK92-FasLmod3 cells showed the highest cytotoxicity against HEK293 comparing to other modified NK92 cells.

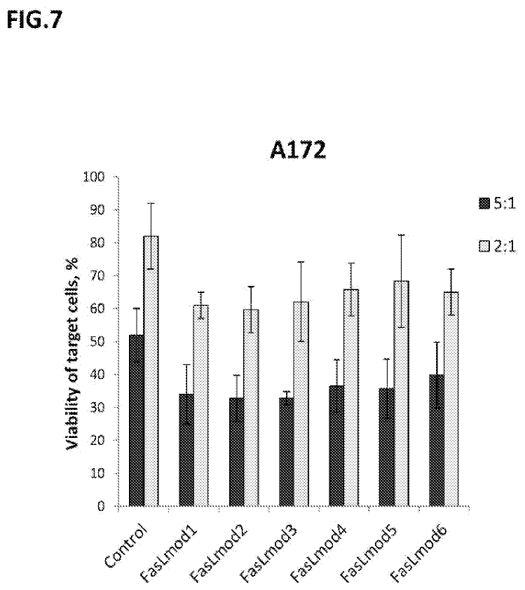

FIG. 7 depicts the cytotoxicity of mock NK92, NK92-FasLmod1, NK92-FasLmod2, NK92-FasLmod3, NK92-FasLmod4, NK92-FasLmod5, NK92-FasLmod6 cells against A172 cells. As with other tumor cells, the modified NK92 cells expressing FasL muteins, exhibited higher killing activity against A172 cells than mock NK92 cells. At the E:T ratio of 2:1, 39%, 40%, 39%, 35%, 31% and 35% of A172 cells were killed by NK92-FasLmod1, NK92-FasLmod2, NK92-FasLmod3, NK92-FasLmod4, NK92-FasLmod5, NK92-FasLmod6 cells, respectively. The mock NK-92 cytotoxicity against A172 cells was 18% at this ratio. At the E:T ratio of 5:1, 65%, 68%, 68%, 68%, 67% and 60% of A172 cells were killed by NK92-FasLmod1, NK92-FasLmod2, NK92-FasLmod3, NK92-FasLmod4, NK92-FasLmod5, NK92-FasLmod6 cells, respectively. The mock NK-92 cytotoxicity against A172 cells was 48% at this ratio. Note that against A172 cells, the cytotoxic activity of the various modified TL92 cells was about the same and did not differ as much as against the other tumor target cells described above (HeLa and HEK293 cells).

Noteworthy, these data show that even at a modest E:T ratio (2:1), the modified NK cells exhibit high cytotoxic activity. This suggests that the desirable cytotoxic effects of modified NK cells expressing FasL muteins can be realized even when NK cells are present in moderate numbers relative to the target cells, which is likely to be the case in clinical applications. Moreover, these data show that the modified NK cells disclosed herein have significantly increased cytotoxicity compared to unmodified NK cells.

### Example 4—Comparative Assessment of the Proliferation Rate of NK92 and NK92-FasLmod1, NK92-FasLmod2, NK92-FasLmod3, NK92-FasLmod4, NK92-FasLmod5, NK92-FasLmod6 Cell Cultures

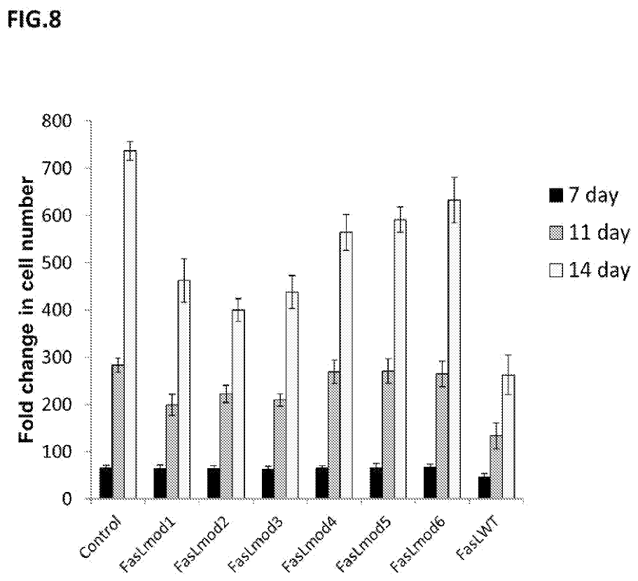

Cell proliferation rate was assessed by trypan blue staining and light microscopic quantification of live cells at different time points (63). NK92 cells, NK92-FasLmod1, NK92-FasLmod2, NK92-FasLmod3, NK92-FasLmod4, NK92-FasLmod5 and NK92-FasLmod6 were seeded (0.5×106 cells/mL) in αMEM medium with 2 mM L-glutamine, sodium bicarbonate (1.5 g/L), 0.2 mM inositol, 0.1 mM 2-mercaptoethanol, 0.02 mM folic acid, 400 U/ml recombinant interleukin 2, 25% fetal calf serum and incubated for 14 days at 370 C and 5% CO2. Samples for analysis were taken on days 7, 11 and 14. All experiments were performed in triplicate, and data are expressed as the mean of three samples with standard deviation. The results of proliferative activity assessment are shown in FIG. 8.

In all groups, cell proliferation steadily increased up to the 14th day. However, as early as day 11, it was noticeable that the fold change of NK92 cells transfected with wild-type FasL was significantly lower than that of control NK92 cells and NK92 cells transfected with FasL muteins. And by day 14, the difference with control NK92, NK92-FasLmod1, NK92-FasLmod2, NK92-FasLmod3, NK92-FasLmod4, NK92-FasLmod5, NK92-FasLmod6 cells was 2.5, 1.6, 1.4, 1.5, 1.9, 2.0, and 2.1 times, respectively. The fold change of NK92-FasLmod1, NK92-FasLmod2, NK92-FasLmod3 cells was slightly lower than that of NK92-FasLmod4, NK92-FasLmod5 or NK92-FasLmod6 cells. The difference between NK92-FasLmod1, NK92-FasLmod2, NK92-FasLmod3 cells and NK92-FasLmod4, NK92-FasLmod5, NK92-FasLmod6 cells in the levels of survival and cytotoxicity is apparently determined by the presence of additional “trafficking domains” in the intracellular part of NK92-FasLmod4, NK92-FasLmod5, NK92-FasLmod6 cells. Thus, according to some of the embodiments disclosed here, these data indicate that, in contrast to wild-type FasL, overexpression of FasL muteins promotes the survival of transfected cells and enhances their cytotoxic activity.

### Example 5—Effect of a Truncated Promoter, ATRA, and Vitamin E on the Level of Fas Ligand Expression and Survival of Modified NK92 Cells

NK92 cells were transfected with a plasmid vector encoding the recombinant FasLmod1 gene with the natural fasIg promoter or a truncated fasIg promoter or cytomegalovirus promoter. After selection in the medium with G418, the expression level of the recombinant gene was assessed by polymerase chain reaction coupled with reverse transcription (RT-PCR) using the primers ExF (SEQ ID NO: 45) and ExR (SEQ ID NO: 46).

Two-step RT-PCR reactions were performed using RevertAid M-MuLV reverse transcriptase (Thermo Scientific, USA) and Taq DNA polymerase (Thermo Scientific, USA) in accordance with the manufacturer's recommendations. 5 μg of RNA was combined with 0.5 ng of oligo dT12-18 mer and incubated in water at 65 °C for 5 min followed by incubation on ice. First strand synthesis was performed in buffer containing 50 mM Tris-HCl (pH 8.3 at 25 °C), 50 mM KCl, 4 mM MgCl₂, 10 mM DTT, 1 mM dNTPs, 10 U/μl RevertAid M-MuLV reverse transcriptase for 60 min at 42 °C. The reaction was terminated by heating at 70 °C for 10 min. A fifth of the reaction was used as a template in a reaction volume of 25 μl containing 10 mM Tris-HCl (pH 8.8 at 25 °C), 50 mM KCl, 0.08% (v/v) Nonidet P40, 2 mM MgCl₂, 0.2 mM dNTPs (Thermo Scientific, USA), 0.2 μM of the primers and 0.6 U of Taq DNA polymerase (Thermo Scientific, USA). The amplification protocol was as follow: an initial denaturation at 95 °C for 10 min, followed by 30 cycles at 95 °C for 30 s, 59 °C for 30 s, 72 °C for 30 s, and a final extension at 72 °C for 10 min. The size of each amplification product was resolved by electrophoresis in a 1.2% agarose gel (w/v) prepared in TAE buffer (40 mM Tris, pH 8.3, 20 mM acetic acid, 1 mM EDTA) with 0.4 μg/ml ethidium bromide. 6×DNA Loading Dye containing 10 mM Tris-HCl (pH 7.6) 0.03% bromophenol blue, 0.03% xylene cyanol FF, 60% glycerol, 60 mM EDTA was used for loading DNA markers and samples. GeneRuler 100 bp DNA Ladder (Thermo Scientific, USA) was used as molecular weight standard. The results are shown in FIG. 9A. The activity of the truncated promoter is higher than that of the natural promoter, but lower than that of the commonly used CMV promoter. These data demonstrate that, in accordance with several embodiments disclosed herein, the use of this truncated promoter makes it possible to achieve a significantly higher level of Fas ligand expression compared to the natural one.

Cell proliferation rate was assessed by trypan blue staining and light microscopic quantification of live cells at different time points (Strober. Curr Protoc Immunol. 2015 Nov. 2; 111: A3.B.1-A3.B.3). Transfected cells were seeded (0.5×10⁶ cells/mL) in αMEM medium with 2 mM L-glutamine, sodium bicarbonate (1.5 g/L), 0.2 mM inositol, 0.1 mM 2-mercaptoethanol, 0.02 mM folic acid, 400 U/ml recombinant interleukin 2, 25% fetal calf serum and incubated for 15 days at 37 °C and 5% CO2. Samples for analysis were taken on days 7, 11 and 15. All experiments were performed in triplicate, and data are expressed as the mean of three samples with standard deviation. The results of proliferative activity assessment are shown in FIG. 9B. Expression of the Fas ligand under the control of the truncated hfasIg promoter ensures greater survival of NK92 cells than under the action of the CMV promoter. According to some embodiments disclosed herein, a truncated version of the hfasIg promoter can be incorporated into an expression vector encoding modified forms of FasL to enhance Fas ligand expression within cells and preserve cell viability.

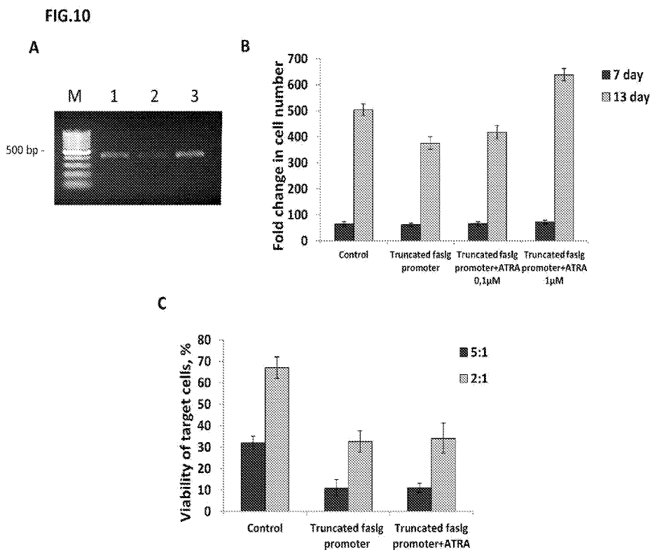

The effects of ATRA on the expression level of Fas ligand and survival of modified NK92 cells were assessed as follows. NK92-FasLmod1 with a truncated fasIg promoter was incubated for 72 h in the presence of 1 μM ATRA or without it. RNA was isolated from the cells, and the expression level of FasL was assessed by polymerase chain reaction coupled with reverse transcription (RT-PCR) using the primers ExF (SEQ ID NO: 45) and ExR (SEQ ID NO: 46) as described above. The results are shown in FIG. 10A.

Cell proliferation rate was assessed by trypan blue staining and light microscopic quantification of live cells at different time points (63). Control NK92 cells and NK92-FasLmod1 cells with a truncated fasIg promoter were seeded (0.5×10⁶ cells/mL) in αMEM medium with 2 mM L-glutamine, sodium bicarbonate (1.5 g/L), 0.2 mM inositol, 0.1 mM 2-mercaptoethanol, 0.02 mM folic acid, 400 U/ml recombinant interleukin 2, 25% fetal calf serum and incubated for 72 h in the presence of 1 μM, or 0.1 μM ATRA or without it. After it cells were incubated for 13 days. Samples for analysis were taken on days 7, and 13. All experiments were performed in triplicate, and data are expressed as the mean of three samples with standard deviation. The assessment of a fold change in cell number is shown in FIG. 10B.

To evaluate the effect of ATRA on the cytotoxic activity of control NK92 cells and NK92-FasLmod1 cells with a truncated fasIg promoter, a cytotoxicity assay was performed using Hela cells as a target. Modified cells were incubated in medium with 1 μM ATRA for 13 days, then replaced with medium without ATRA and cultured for additional 48 h. Then cells were added to the wells of the 96-well plate with the previously seeded target Hela cells and incubated for 5 h at 37 °C and 5% CO₂ at a ratio of 2:1 or 5:1 (effector:target). The wells were then washed with buffered saline and the number of surviving target cells was estimated by staining with neutral red dye. Data summarizing the percent cytotoxicity of NK92 cells and NK92-FasLmod1 cells preincubated with ATRA are shown in FIG. 10C.

These data demonstrate that, in accordance with several embodiments of the invention disclosed here, the use of a truncated promoter and the addition of ATRA during cultivation leads to suppression of FasL expression during in vitro cell cultivation. As a result, the viability of the modified NK cells and the yield of the cell product at the end of the cultivation cycle are increased. After removal of ATRA at the final stage of cultivation, the expression level of FasL and the cytotoxic activity of cells are restored. Thus, the use of a truncated promoter for the expression of modified FasL forms in combination with ATRA makes it possible to obtain a greater yield of NK cells with increased survival and high cytotoxic activity.

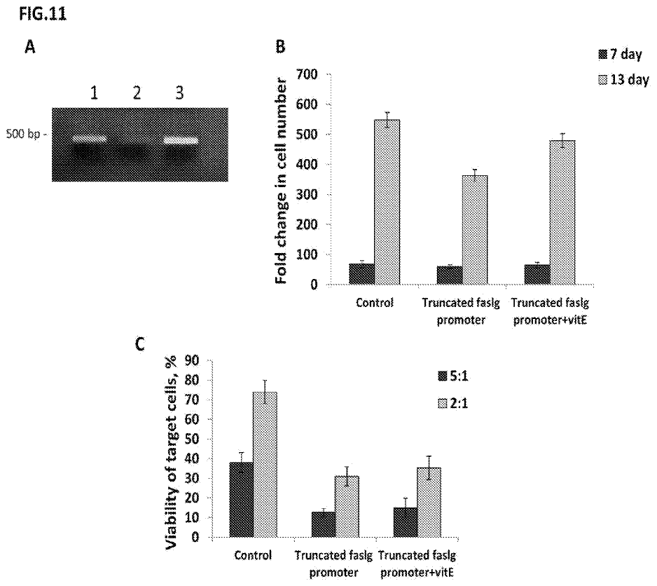

The effects of vitamin E on the expression level of Fas ligand and survival of modified NK92 cells were assessed as follows. NK92-FasLmod1 with a truncated fasIg promoter was incubated for 4 h in the presence of 40 μM vitamin E or without it. RNA was isolated from the cells, and the expression level of FasL was assessed by polymerase chain reaction coupled with reverse transcription (RT-PCR) using the primers ExF (SEQ ID NO: 45) and ExR (SEQ ID NO: 46) as described above. The results are shown in FIG. 11A.

Cell proliferation rate was assessed by trypan blue staining and light microscopic quantification of live cells at different time points. Control NK92 cells and NK92-FasLmod1 cells with a truncated fasIg promoter were seeded (0.5×10⁶ cells/mL) in αMEM medium with 2 mM L-glutamine, sodium bicarbonate (1.5 g/L), 0.2 mM inositol, 0.1 mM 2-mercaptoethanol, 0.02 mM folic acid, 400 U/ml recombinant interleukin 2, 25% fetal calf serum and incubated for 13 days in the presence of 25 μM vitamin E or without it. Samples for analysis were taken on days 7, and 13. All experiments were performed in triplicate, and data are expressed as the mean of three samples with standard deviation. The assessment of a fold change in cell number is shown in FIG. 11B.

To evaluate the effect of vitamin E on the cytotoxic activity of control NK92 cells and NK92-FasLmod1 cells with a truncated fasIg promoter, a cytotoxicity assay was performed using Hela cells as a target. Modified cells were incubated in medium with 40 μM vitamin E for 4 h, then replaced with medium without vitamin E and cultured for additional 24 h. Then cells were added to the wells of the 96-well plate with the previously seeded target Hela cells and incubated for 5 h at 37 °C and 5% CO2 at a ratio of 2:1 or 5:1 (effector:target). The wells were then washed with buffered saline and the number of surviving target cells was estimated by staining with neutral red dye (62). Data summarizing the percent cytotoxicity of NK92 cells and NK92-FasLmod1 cells preincubated with vitamin E are shown in FIG. 11C.

These data demonstrate that, in accordance with several embodiments of the invention disclosed here, the use of a truncated promoter and the addition of ATRA during cultivation leads to suppression of FasL expression during in vitro cell cultivation. As a result, the viability of the modified NK cells and the yield of the cell product at the end of the cultivation cycle are increased. After removal of ATRA at the final stage of cultivation, the expression level of FasL and the cytotoxic activity of cells are restored. Thus, the use of a truncated promoter for the expression of modified FasL forms in combination with ATRA makes it possible to obtain a greater yield of NK cells with increased survival and high cytotoxic activity.

In accordance with some embodiments, an additional positive effect in obtaining modified cells can be achieved by adding vitamin E and its derivatives when culturing modified NK cells in vitro. In some embodiments of the invention, the compositions with the use of a truncated promoter and the addition of vitamin E during cultivation leads to the suppression of FasL expression during cell production in vitro. As a result, the viability of the modified NK cells and the yield of the cell product at the end of the cycle of cultivation are increased. After removal of vitamin E at the final stage of cultivation, the expression level of FasL and the cytotoxic activity of cells are restored. Thus, the use of a truncated promoter for the expression of modified FasL forms in combination with vitamin E makes it possible to obtain a greater yield of NK cells with increased survival and high cytotoxic activity.

Thus, the present disclosure provides the Fas ligand variant, the recombinant cell, such as the recombinant NK cell, the pharmaceutical composition, the vector providing the genetic material for producing the FasL variant in the recombinant cell and the methods of their production, as well as FasL variant for use in the therapy of immune related diseases.

### REFERENCE TO SEQUENCE LISTING

This application contains a Sequence Listing submitted as an electronic ST26 XML file and is hereby incorporated by reference in its entirety.

1. A Fas Ligand (FasL) variant having an intracellular domain comprising at least one amino acid sequence GYXXΦ, wherein (X) is any amino acid, and (Φ) is an amino acid selected from an amino acid L, I or V, wherein an amino acid sequence of the FasL variant has at least 80% sequence identity with the amino acids 1-80 of the SEQ ID NO: 1, and wherein the amino acid positions of the amino acid sequence of the FasL variant correspond to the amino acid positions of the SEQ ID NO: 1.

2. The FasL variant of claim 1, wherein the intracellular domain comprises an amino acid sequence ⁶GYGYΦQIYWVZ¹⁶, wherein both, (Φ) and (Z), are selected independently from an amino acid L, I or V, and wherein the amino acid positions of the amino acid sequence correspond to the amino acid positions of the SEQ ID NO: 1.

3. The FasL variant of claim 1, wherein the intracellular domain comprises an amino acids ⁶GYGYLQIYWVL¹⁶, or ⁶GYGYIQIYWVI¹⁶, or ⁶GYGYVQIYWVV¹⁶, or ⁶GYGYLQIYWVL¹⁶ and ⁶⁷GYPPL⁷¹, or ⁶GYGYIQIYWVI¹⁶ and ⁶⁷GYPPI⁷¹, or ⁶GYGYVQIYWVV¹⁶ and ⁶⁷GYPPV⁷¹, and wherein the amino acid positions correspond to the amino acid positions of the SEQ ID NO: 1.

4. The FasL variant of claims 1-3, comprising an amino acid sequence selected from any one of SEQ ID NO: 1 to SEQ ID NO: 6.

5. The FasL variant of claim 1, comprising at least one amino acid substitution when compared to a wild type FasL, and wherein the amino acid substitution is configured to facilitate transportation of the FasL variant to secretory lysosomes, when expressed in a cell.

6. A recombinant cell comprising genetic elements that allow producing at least one FasL variant of claim 1.

7. The recombinant cell of claim 6, wherein the recombinant cell is a human Natural Killer (NK) cell.

8. A vector comprising a polynucleotide encoding the FasL variant of claim 1 and a FasL promoter.

9. The vector according to the claim 8, wherein the FasL promoter of the vector is a truncated FasL promoter, preferably the truncated promoter has a nucleotide sequence of SEQ ID NO: 13.

10. A pharmaceutical composition comprising the recombinant cell of claim 6 and at least one further component selected from a pharmaceutically acceptable excipient, carrier, and/or adjuvant.

11.-15. (canceled)

16. A method of treating a medical condition in an individual in need thereof, comprising administering to the individual a therapeutically effective amount of natural killer (NK) cells modified to express the Fas ligand (FasL) variant of claim 1, wherein the NK cells are configured to have increased cytotoxicity and survival compared to NK cells producing unmodified FasL.

17. The method of claim 16, wherein the NK cells are autologous or allogeneic with respect to the individual.

18. The method of claim 16, wherein the NK cells are derived from umbilical cord blood, peripheral blood, bone marrow, cells infiltrated into tissues, CD34+ cells, induced pluripotent stem cells (iPSCs), embryonic stem cells (ESCs), or a human NK cell line.

19. The method of claim 16, wherein said NK cells expressing FasL variants are cultured, expanded, activated or stimulated prior to administration to the individual.

20. The method of claim 16, wherein said NK cells expressing FasL variants are cultured, expanded, activated or stimulated with various concentrations of all-trans-retinoic acid (ATRA) or with vitamin E or its derivatives, prior to administration to the individual.

21. The FasL variant of claim 1, wherein an amino acid sequence of the FasL variant has at least 85% sequence identity with the amino acids 1-80 of the SEQ ID NO: 1.

22. The FasL variant of claim 1, wherein an amino acid sequence of the FasL variant has at least 90% sequence identity with the amino acids 1-80 of the SEQ ID NO: 1.

23. The FasL variant of claim 1, wherein herein an amino acid sequence of the FasL variant has at least 95% sequence identity with the amino acids 1-80 of the SEQ ID NO: 1.

24. The FasL variant of claim 1, wherein an amino acid sequence of the FasL variant has at least 97% sequence identity with the amino acids 1-80 of the SEQ ID NO: 1.

25. The FasL variant of claim 2, wherein the intracellular domain comprises an amino acids ⁶GYGYLQIYWVL¹⁶, or ⁶GYGYIQIYWVI¹⁶, or ⁶GYGYVQIYWVV¹⁶, or ⁶GYGYLQIYWVL¹⁶ and ⁶⁷GYPPL⁷¹, or ⁶GYGYIQIYWVI¹⁶ and ⁶⁷GYPPI⁷¹, or ⁶GYGYVQIYWVV¹⁶ and ⁶⁷GYPPV⁷¹, and wherein the amino acid positions correspond to the amino acid positions of the SEQ ID NO: 1.


SEQUENCE LISTING (47 sequences)

SEQ ID NO: 1 - protein, 281 aa, synthetic construct
MQQPFGYGYLQIYWVLSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 2 - protein, 281 aa, synthetic construct
MQQPFGYGYIQIYWVISSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 3 - protein, 281 aa, synthetic construct
MQQPFGYGYVQIYWVVSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 4 - protein, 281 aa, synthetic construct
MQQPFGYGYLQIYWVLSSASSPWAPPGTVLPCPTSVPRRPGQRRPPPPPPPPPLPPPPPPPPLPPLGYPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 5 - protein, 281 aa, synthetic construct
MQQPFGYGYIQIYWVISSASSPWAPPGTVLPCPTSVPRRPGQRRPPPPPPPPPLPPPPPPPPLPPLGYPPIKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 6 - protein, 281 aa, synthetic construct
MQQPFGYGYVQIYWVVSSASSPWAPPGTVLPCPTSVPRRPGQRRPPPPPPPPPLPPPPPPPPLPPLGYPPVKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 7 - DNA, 846 bp, synthetic construct
  source 1..846: mol_type = other DNA
atgcagcagcccttcggctacggctatctgcagatctactgggtgctgagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaa

SEQ ID NO: 8 - DNA, 846 bp, synthetic construct
  source 1..846: mol_type = other DNA
atgcagcagcccttcggctacggctatatccagatctactgggtgatcagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaa

SEQ ID NO: 9 - DNA, 846 bp, synthetic construct
  source 1..846: mol_type = other DNA
atgcagcagcccttcggctacggctatgtgcagatctactgggtggtgagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaa

SEQ ID NO: 10 - DNA, 846 bp, synthetic construct
  source 1..846: mol_type = other DNA
atgcagcagcccttcggctacggctatctgcagatctactgggtgctgagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaggctacccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaa

SEQ ID NO: 11 - DNA, 846 bp, synthetic construct
  source 1..846: mol_type = other DNA
atgcagcagcccttcggctacggctatatccagatctactgggtgatcagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaggctacccacccatcaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaa

SEQ ID NO: 12 - DNA, 846 bp, synthetic construct
  source 1..846: mol_type = other DNA
atgcagcagcccttcggctacggctatgtgcagatctactgggtggtgagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaggctacccacccgtgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaa

SEQ ID NO: 13 - DNA, 453 bp, Homo sapiens
  source 1..453: mol_type = genomic DNA
ttatagccccactgaccattctcctgtagctgggagcagttcacactaacagggctatacccccatgctgacctgctctgcaggatcccaggaaggtgagcatagcctactaacctgtttgggtagcacagcgacagcaactgaggccttgaaggctgttatcagaaaattgtgggcggaaacttccaggggtttgctctgagcttcttgaggcttctcagcttcagctgcaaagtgagtgggtgtttctttgagaagcagaatcagagagagagagatagagaaagagaaagacagaggtgtttcccttagctatggaaactctataagagagatccagcttgcctcctcttgagcagtcagcaacagggtcccgtccttgacacctcagcctctacaggactgagaagaagtaaaaccgtttgctggggctggcctgactcaccagctgcc

SEQ ID NO: 14 - DNA, 32 bp, Homo sapiens
  source 1..32: mol_type = other DNA
ttatagccccactgaccattctcctgtagctg

SEQ ID NO: 15 - DNA, 25 bp, Homo sapiens
  source 1..25: mol_type = other DNA
ctgcatggcagctggtgagtcaggc

SEQ ID NO: 16 - DNA, 40 bp, synthetic construct
  source 1..40: mol_type = other DNA
atgcagcagcccttcggctacggctatctgcagatctact

SEQ ID NO: 17 - DNA, 40 bp, synthetic construct
  source 1..40: mol_type = other DNA
atgcagcagcccttcggctacggctatatccagatctact

SEQ ID NO: 18 - DNA, 40 bp, synthetic construct
  source 1..40: mol_type = other DNA
atgcagcagcccttcggctacggctatgtgcagatctact

SEQ ID NO: 19 - DNA, 40 bp, synthetic construct
  source 1..40: mol_type = other DNA
gggtgctgagcagtgccagctctccctgggcccctccagg

SEQ ID NO: 20 - DNA, 40 bp, synthetic construct
  source 1..40: mol_type = other DNA
gggtgatcagcagtgccagctctccctgggcccctccagg

SEQ ID NO: 21 - DNA, 40 bp, synthetic construct
  source 1..40: mol_type = other DNA
gggtggtgagcagtgccagctctccctgggcccctccagg

SEQ ID NO: 22 - DNA, 40 bp, Homo sapiens
  source 1..40: mol_type = other DNA
cacagttcttccctgtccaacctctgtgcccagaaggcct

SEQ ID NO: 23 - DNA, 40 bp, Homo sapiens
  source 1..40: mol_type = other DNA
ggtcaaaggaggccaccaccaccaccgccaccgccaccac

SEQ ID NO: 24 - DNA, 38 bp, Homo sapiens
  source 1..38: mol_type = other DNA
taccacctccgccgccgccgccaccactgcctccacta

SEQ ID NO: 25 - DNA, 40 bp, Homo sapiens
  source 1..40: mol_type = other DNA
ccgctgccacccctgaagaagagagggaaccacagcacag

SEQ ID NO: 26 - DNA, 40 bp, synthetic construct
  source 1..40: mol_type = other DNA
ggctacccacccctgaagaagagagggaaccacagcacag

SEQ ID NO: 27 - DNA, 40 bp, synthetic construct
  source 1..40: mol_type = other DNA
ggctacccacccatcaagaagagagggaaccacagcacag

SEQ ID NO: 28 - DNA, 40 bp, synthetic construct
  source 1..40: mol_type = other DNA
ggctacccacccgtgaagaagagagggaaccacagcacag

SEQ ID NO: 29 - DNA, 37 bp, Homo sapiens
  source 1..37: mol_type = other DNA
ggagacacaggcctgtgctgtggttccctctcttctt

SEQ ID NO: 30 - DNA, 40 bp, Homo sapiens
  source 1..40: mol_type = other DNA
caggggtggcagcggtagtggaggcagtggtggcggcggc

SEQ ID NO: 31 - DNA, 40 bp, synthetic construct
  source 1..40: mol_type = other DNA
caggggtgggtagcctagtggaggcagtggtggcggcggc

SEQ ID NO: 32 - DNA, 40 bp, synthetic construct
  source 1..40: mol_type = other DNA
gatgggtgggtagcctagtggaggcagtggtggcggcggc

SEQ ID NO: 33 - DNA, 40 bp, synthetic construct
  source 1..40: mol_type = other DNA
cacgggtgggtagcctagtggaggcagtggtggcggcggc

SEQ ID NO: 34 - DNA, 40 bp, Homo sapiens
  source 1..40: mol_type = other DNA
ggcggaggtggtagtggtggcggtggcggtggtggtggtg

SEQ ID NO: 35 - DNA, 40 bp, Homo sapiens
  source 1..40: mol_type = other DNA
gcctcctttgaccaggccttctgggcacagaggttggaca

SEQ ID NO: 36 - DNA, 40 bp, Homo sapiens
  source 1..40: mol_type = other DNA
gggaagaactgtgcctggaggggcccagggagagctggca

SEQ ID NO: 37 - DNA, 40 bp, synthetic construct
  source 1..40: mol_type = other DNA
ctgctcagcacccagtagatctgcagatagccgtagccga

SEQ ID NO: 38 - DNA, 40 bp, synthetic construct
  source 1..40: mol_type = other DNA
ctgctgatcacccagtagatctggatatagccgtagccga

SEQ ID NO: 39 - DNA, 40 bp, synthetic construct
  source 1..40: mol_type = other DNA
ctgctcaccacccagtagatctgcacatagccgtagccga

SEQ ID NO: 40 - DNA, 21 bp, Homo sapiens
  source 1..21: mol_type = other DNA
tgccatgcagcagcccttcgg

SEQ ID NO: 41 - DNA, 28 bp, Homo sapiens
  source 1..28: mol_type = other DNA
catcacaaggagacacaggcctgtgctg

SEQ ID NO: 42 - DNA, 28 bp, Homo sapiens
  source 1..28: mol_type = other DNA
cagcacaggcctgtgtctccttgtgatg

SEQ ID NO: 43 - DNA, 37 bp, synthetic construct
  source 1..37: mol_type = other DNA
cacctcgagttagagcttatataagccgaaaaacgtc

SEQ ID NO: 44 - DNA, 31 bp, synthetic construct
  source 1..31: mol_type = other DNA
tttacgcgttatagccccactgaccattctc

SEQ ID NO: 45 - DNA, 27 bp, synthetic construct
  source 1..27: mol_type = other DNA
gagaagcaaataggccacccagtccac

SEQ ID NO: 46 - DNA, 36 bp, synthetic construct
  source 1..36: mol_type = other DNA
gaccttatataagccgaaaaaacgtctgagattctc

SEQ ID NO: 47 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL